(19) United States
(12) Patent Application Publication
(10) Pub. No.: US 2022/0202733 A1
(43) Pub. Date: Jun. 30, 2022

(54) PROTEIN NANO- OR MICROPARTICLES AS ARTIFICIAL INCLUSION BODIES

(71) Applicants: UNIVERSITAT AUTONOMA DE BARCELONA, Bellaterra (ES); INSTITUT RECERCA HOSPITAL DE LA SANTA CREU I SANT PAU, Barcelona (ES); CONSORCIO CENTRO DE INVESTIGACIÓN BIOMÉDICA EN RED, M.P. (CIBER), Madrid (ES)

(72) Inventors: Antonio VILLAVERDE CORRALES, Barcelona (ES); Esther VÁZQUEZ GÓMEZ, Barcelona (ES); Héctor LÓPEZ LAGUNA, Barcelona (ES); Julieta María SÁNCHEZ, Barcelona (ES); Ramón MANGUES BAFALLUY, Barcelona (ES); Patricia ALAMO, Barcelona (ES)

(73) Assignees: UNIVERSITAT AUTONOMA DE BARCELONA, Bellaterra (ES); INSTITUT RECERCA HOSPITAL DE LA SANTA CREU I SANT PAU, Barcelona (ES); CONSORCIO CENTRO DE INVESTIGACIÓN BIOMÉDICA EN RED, Madrid (ES)

(21) Appl. No.: 17/602,402

(22) PCT Filed: Apr. 8, 2020
(86) PCT No.: PCT/EP2020/059994

§ 371 Date: Oct. 8, 2021

(30) Foreign Application Priority Data
Apr. 11, 2019 (EP) 19382271.5
Oct. 17, 2019 (EP) 19382909.0

(51) Int. Cl. A61K 9/51; A61K 9/00; C07K 14/00; A61K 9/16

(52) U.S. Cl. CPC A61K 9/5169; A61K 9/0019; A61K 38/00; A61K 9/1658; C07K 14/001

(57) ABSTRACT

The invention relates to protein particles comprising a cluster of one or more types of assembled self-contained protein, wherein the particle has a size from 50 nm to 50 micrometers; is in form of a pellet in aqueous media; is mechanically stable; and it release a particular percentage by weight of the self-contained protein within a predetermined period of time, and any other compound contained in the particle. Particular methods for obtaining the particles are also disclosed, said methods comprising the addition of salts to allow precipitation of proteins. Particular protein particles comprising lipids associated with the assembly of self-contained proteins are also disclosed. The invention also relates to several uses of the particle, in particular medical uses and to pharmaceutical and cosmetic compositions comprising the particles.

This application claims the benefit of European Patent Application EP19382271, filed Apr. 11, 2019; and of European Patent Application EP19382909.0, filed Oct. 17, 2019.

### TECHNICAL FIELD

Present invention relates to the field of self-assembled biological molecules and to particular uses of the same in the field of medicine.

### BACKGROUND ART

Bacterial inclusion bodies (IBs) are mechanically stable, insoluble, discrete, and particulate proteinaceous materials produced in recombinant bacteria, with particle sizes ranging from aprox. 50 to 1500 nm, and with shapes including cylindrical, amorphous, spherical or ellipsoid. They contain one or few functional protein species (together with other possible components) that can be released under physiological conditions. They commonly occur in the cytoplasm of recombinant bacteria, as a consequence of conformational stresses occurring during protein overproduction. They are mainly formed by the recombinant protein, but also contain variable but uncharacterized amounts of bacterial proteins, lipids carbohydrates and nucleic acids (see Neubauer, et al., “Protein inclusion bodies in recombinant bacteria”, 237-292, Springer-2006). IBs have been traditionally observed as an obstacle to obtain functional protein by recombinant procedures, since insoluble protein was believed to be unfolded and inactive. However, in 2005, inventors and others discovered that IBs are formed by a mixture of non-functional and functional polypeptides, and that IBs are protein particles with biological activity, usable in biotechnology and biomedicine (see. Garcia-Fruitos, E. et al. “Aggregation as bacterial inclusion bodies does not imply inactivation of enzymes and fluorescent proteins”, Microbial cell factories 2005, vol. no. 4, 27, doi:10.1186/1475-2859-4-27; and Gonzalez-Montalban, N., et al., “Recombinant protein solubility—does more mean better?”, Nature biotechnology 2007, vol. no. 25, pp.: 718-720, doi:10.1038/nbt0707-718). The non-functional protein has an amyloidal structure that corresponds to around 20-40% of the bulk material. The functional protein is embedded in this amyloidal structure in a sort of sponge-like organization.

Thus, IBs are mechanically stable functional materials that are nontoxic when exposed to cells or to living beings, through oral administration or injection. Because of the combination of mechanical stability and functionality, IBs are then explored as self-immobilized catalysts, showing promises in biotechnological industries and applications (see Rinas, U. et al. “Bacterial Inclusion Bodies: Discovering Their Better Half”. Trends in biochemical sciences, doi:10.1016/j.tibs.2017.01.005 (2017); and de Marco, A. et al. “Bacterial inclusion bodies are industrially exploitable amyloids”, FEMS microbiology reviews, doi:10.1093/femsre/fuy038 (2018)).

As indicated, when in contact with mammalian cells, IBs tend to penetrate them, apparently in absence of toxicity, and are able to release the embedded functional protein with full biological activity. The cell contact and penetration can be in addition targeted by fusing a cell-targeting peptide to the recombinant IB protein. Functional IBs can then act as NANOPILLS for the delivery of therapeutic proteins. This principle has been demonstrated for chaperones, enzymes, growth factors and structural proteins, and it can be also applied to cytokines, hormones, and any functional protein having a physiological role, whose activity needs to be restored or enhanced. This can be also used by de novo designed proteins with activities or combination of them, not present in nature. When administered by local injection in tumor or subcutaneously, IBs are stable in the injection site and slowly release the IB protein for a functional effect, either locally or remotely, when the protein is targeted with a homing peptide. When the IB protein self-assembled as tumor-targeted nanoparticles, they released from IBs in the assembled form, both in vitro and in vivo. Then, subcutaneous injection in a remote place provides a long-lasting depot platform for the delivery of tissue-targeted therapeutic protein nanoparticles for diverse clinical applications.

As catalysts, IBs do not pose any regulatory issues and are highly convenient. However, clinical applicability of IBs is not exempt of drawbacks and for this reason it is restricted by the following facts:

First, IBs have a bacterial origin, and they contain irremovable bacterial components at variable composition such as cell wall, nucleic acids, endotoxins and undesired proteins, incompatible with a drug formulation. Moreover, due to the cell factory base, IBs carry on with several homogeneity issues between manufacturing batches.

Secondly, proteins that glycosylate or that follow other post-translational modifications not done by bacterial cells cannot be produced as functional IBs.

Finally, and as previously enunciated, although apparently non-toxic even having an amyloid structure, few assays and data are still in relation thereto.

For all these reasons, the provision of alternative structures for delivering compounds of interest (small molecule drugs, therapeutic proteins, enzymes, etc.) in cells of organisms is evidence, but the maintenance of all those beneficial behaviour and features of IBs is also a desire. In particular, their high penetration to cells and the mechanic stability.

### SUMMARY OF INVENTION

Inventors developed insoluble protein particles, with a slow release profile of the proteins at physiological conditions comprising clusters or groupings of assembled self-contained proteins that surprisingly mimic the goodness of IBs but that are exempts of the drawbacks. Artificial IBs have been prepared in vitro (cell-free engineered) without the presence of bacterial cells, thus in a fully synthetic mode. According to the best of inventor's knowledge this is the first time protein micro and nanoparticles have been done resembling the natural IBs, avoiding furthermore by this way the problematic features existing in natural IBs as described before.

Thus, a first aspect of the invention is a protein nano- or microparticle comprising a cluster of one or more types of assembled self-contained proteins, and one or more salts of divalent cations, being the ratio of moles of salt of divalent cation:moles of self-contained protein comprised from 4:1 to 1000:1, wherein the nano- or micro particle:
has a size, measured as hydrodynamic diameter, from 50 nanometers (nm) to 50 micrometers (μm);
is mechanically stable, which means that the cluster of self-contained proteins remains structured when submitted at sonication conditions including 5 rounds of 40 seconds; 0.5 of pulse on; 0.5 of pulse off and a wave width of 10% in a high intensity sonicator Branson sonifier 450, with 3 mm-diameter titanium probe;
is in the form of a precipitated pellet in aqueous media, when centrifuged at 15.000 g at a temperature from 4° C. to 30° C.; and
it releases an amount of assembled self-contained proteins lower than 50% by weight in relation to the total weight of assembled self-contained protein within 24 hours and when resuspended in an aqueous media at physiological temperature.

Thus, the nanoparticle or microparticle is obtained as an insoluble pellet, said solubility measured at a temperature from 4° C. to 30° C. (or at room temperature from 18° C. to 30° C.) in an aqueous media.

Sonifier used to test mechanical stability transforms altern current (AC) high voltage to 20 kHz of electrical energy. This high-frequency energy is fed to a converter to obtain mechanical vibration.

Thus, there has been developed synthetic or cell-free engineered protein nano- or microparticles that behave as IBs. For synthetic or cell-free engineered is to be understood that no prokaryotic or eukaryotic cells are the providers/suppliers of said nano- and/or microparticles, but that they are synthesized in a recipient. The cluster or grouping of one or more types of assembled self-contained proteins are indeed configuring a protein scaffold. It is a tridimensional scaffold in which other compounds different from the self—contained proteins can be adhered or embedded between interstitial spaces defined by the assembled proteins, like a net in which other components can be entrapped, and/or adsorbed. This scaffold remains structured once submitted to sonication conditions.

Therefore, the protein nano- or microparticle are free of prokaryotic or eukaryotic cell components different from the self-contained proteins.

As will be illustrated in Examples below, the protein particles of the invention do mimic the properties of natural IBs. Namely, they are mechanically stable; they have a size from 50 nm to 50 μm; they are formed by proteins that are self-contained and they optionally comprise additional functional proteins. In certain particular embodiments they are formed by one or more protein species (obtained from an external source, such as a protein supplier), plus any necessary additional components (such as lipids) at a defined proportion. They allow the release of the proteins, either the ones assembled and self-contained or any other protein that is embedded in the cluster of assembled self-contained proteins; and they penetrate mammalian cells in absence of toxicity and release, intracellularly, functional protein. In addition, cell penetrability is targetable.

The design and fabrication of artificial IBs has been approached in the laboratory by means of biophysical proteomic methods as described in the detailed description.

Thus, a second aspect of the invention is a method for the synthesis of a protein nano- or microparticle as defined above, wherein the method comprises the following steps:

(a) mixing in a recipient one or more types of proteins with a polar solvent;
(b) submitting the mixture of step (a) to protein assembly conditions to obtain a cluster of assembled self-contained proteins; and
(c) isolating the clusters.

This second aspect is thus a cell-free method for the synthesis of a protein nano- or microparticle.

The invention also relates to films comprising one or more lipids and one or more denatured proteins, which are self-assembled and configure a tridimensional cluster or net, said film disposed on a support.

As will be illustrated in the examples below, the protein nano- or microparticles of the invention penetrate into tumor cells. Thus, a third aspect of the invention is a protein nano- or microparticle as defined above for use as a medicament.

These protein nano- or microparticles resembling natural inclusion bodies are thus, synthetic inclusion bodies, and it is another aspect of the invention a protein nano- or microparticle as defined above as synthetic bacterial inclusion body.

The protein nano- or microparticles comprising functional proteins may, in addition, be used as subcutaneous implants. By this way they can deliver to organisms the compounds with therapeutic effect. These compounds are either the self-contained proteins configuring the cluster of the particle that is sustainly solubilized or decomposed within a period of time, said self-contained proteins having a therapeutic effect, or any therapeutic agent different of the protein that can be embedded or adhered to the cluster of the particles or even linked to the self-contained proteins with an hydrolysable bond. All these options are disclosed in more detail below.

Therefore, another aspect of the invention is a drug-delivery system comprising the protein nano- or microparticles as defined above. For “drug” is to be understood any compound or even composition with a proved therapeutic effect.

The protein nano- or microparticles of the invention can be used as actives in pharmaceutical compositions and so, it is also an aspect of the invention a pharmaceutical composition comprising a therapeutically effective amount of the protein nano- or microparticles disclosed above together with pharmaceutically acceptable excipients or carriers.

Also depending on the nature and functionality (or activity) of the proteins, the protein nano- or microparticles of the invention can be used as actives in cosmetic compositions and so, it is also an aspect of the invention a cosmetic composition comprising a cosmetic effective amount of the protein nano- or microparticles disclosed above together with one or more appropriate cosmetically acceptable excipients or carriers.

### BRIEF DESCRIPTION OF DRAWINGS

FIG. 1, related with Example 2, is a graphic with the detected diameter (calculated by DLS) and the relative abundance of these diameters (% volume) of the protein nano- or microparticles of the invention.

FIG. 2, related with Example 2, shows Field Emission Scanning Electron Microscopy (FESEM) images of synthetic IBs of the invention. Electron high tension, (EHT)=1.00 kV; working distance (WD)=3.3 mm; Signal A=secondary electron (SE2); Mag=2.56 K X.

FIG. 3, related with Example 3, shows fluorescence emission relative intensity measured in a Cary Eclipse spectrofluorimeter (Agilent Technologies, Mulgrave, Australia), at a wavelength of 512 nm] of different concentrations of functional T22-GFP-H6 (g/l) synthetic IBs (light circles) of Example 1. As control nanoparticles (NPs; dark circles) of T22-GFP-H6 formed through assembly of the GFP-containing monomers produced in E. coli.

FIG. 4, related with Example 4, is a graph with the amount of released functional protein in % (i.e. release of functional fluorescent T22-GFP-H6) along time (Time in hours (h)).

FIG. 5 is a graph with the detected intracellular fluorescence in HeLa cells exposed to different entities, analyzed by flow cytometry at different time points. Different assays of internalization were performed using different entities for internalization.

FIG. 6 is a graphic with the percentage of cell viability (% cell viability) at 24 (left column), 48 (middle column) and 72 (right column) hours of cells (HeLa) cultured with the presence of different entities.

FIG. 7, related with Example 7, includes FESEM images of several protein microparticles obtained by assembly of the fused protein T22-GFP-H6 with a salt of divalent cations at different salt:protein molecular proportions (images 1, 2 and 3 for proportions 40:1, 100:1 and 150:1, respectively). They are taken in comparison with a natural inclusion body produced directly in bacteria (image 0).

FIG. 8, related with Example 7, depicts a particular mode of fabrication of ArtIBs of the invention. In FIG. 8 (A) Multiple (ms) and single (ss) step procedures for ArtIB fabrication from soluble pure protein are summarized, indicating the main operational steps (arrows). OS is organic solvent. Precise details can be found in the Methods section of Example 8. Final products are framed. In FIG. 8 (B) Representative FESEM images of alkaline phosphatase (AP) and β-galactosidase (β-Gal) ArtIBs. Magnifications are equivalent in all images. In FIG. 8 (C) DLS size analyses of ArtIBs, indicating the mode (in nm) and the polydispersion index (pdi). In FIG. 8 (D) Specific activity of both AP and β-Gal ArtIBs, compared to that of commercial soluble protein counterpart (first column corresponds to soluble protein, second column corresponds to msArtIBs, and third column corresponds to ssArtIBs). Asterisks indicate statistically different to the specific activity of the soluble protein (Holme-Sidak test, p <0.001).

FIG. 9, also relating to Example 8, shows the characterization of functionally complex ArtIBs. In FIG. 9 (A) FESEM images of CXCR4-targeted ArtIBs, all recorded at the same magnification. At the bottom of each image, specific fluorescence decay (SFD), hydrodynamic size peak (pdi±sem) and percentage of ALS are shown. In FIG. 9 (B) Internalization of T22-GFP-H6 ArtIBs in cultured HeLa cells, recorded at different times after exposure through intracellular GFP fluorescence (top). At bottom, AMD3100-mediated inhibition of ArtIB internalization (columns with dashed pattern in presence of inhibitor; plain columns without inhibitor). In FIG. 9 (C) Viability of cultured HeLa cells upon 96 h of T22-GFP-H6 and T22-PE24-H6 ArtIB exposure in presence (columns with dashed pattern) or absence of AMD3100 (plain columns). In FIG. 9 (D) Stain-free protein detection of released soluble protein (r) from ArtIBs, 7 days after incubation in physiological buffer. In the plot, kinetics of soluble protein release from T22-GFP-H6 ssArtIBs. In FIG. 9 (E) Hydrodynamic mode size peak of T22-GFP-H6 nanoparticles released from ssArtIBs, compared to equivalent soluble nanoparticles after purification from recombinant bacteria (those used for ArtIB fabrication). In the inset, a FESEM image of those nanoparticles released from ArtIBs. In FIG. 9 (F) AMD3100-mediated inhibition of HeLa cell internalization of recombinant soluble and ArtIBs-released nanoparticles. Symbols indicate significant differences to the control (*, p<0.05, Tukey test) and between samples with or without AMD3100 (⁻, p<0.05, Two tail, t-test).

FIG. 10, related with Example 9, depicts ArtIBs material release, tumor uptake and antitumor activity in a CXCR4⁺ colorectal cancer model. In FIG. 10 (A) Preliminary screening of released material and tumor uptake (squares in graphics) after subcutaneous implantation of T22-GFP-H6 msArtIBs, T22-GFP-H6 ssArtIBs (Zn²⁺ 100:1) or PBS (buffer); remaining material at injection point (IP) in diamonds at graphics. In FIG. 10 (B) Representative FLI images obtained at the injection point (IP) and at the remote tumor (T), along time (day 0, 3, 6 and 10) after T22-GFP-H6 ssArtIBs Ca²⁺, T22-GFP-H6 ssArtIBs Zn²⁺ (1:50) or buffer SC administration. In FIG. 10 (C) Antitumor effect, measured as bioluminiscence emission by cancer cells along time, in the CXCR4⁺ SW1417-luci tumor model, after SC injection of 1 mg dose/mouse of T22-GFP-H6 Ca²⁺ ArtIBs, T22-PE24-H6 Ca²⁺ ArtIBs or control PBS (*, p<0.05, Tukey test). Fluorescence (panels a and b) or bioluminescence (panel c) intensity were measured using IVIS® Spectrum and expressed as x̅±SE of average radiant efficiency.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition.

The term “self-contained protein” relates to proteins that when assembled with other proteins constitute by themselves a free-standing unit (cluster) or that remain configuring a certainly ordered scaffold or structure or grouping made of proteins. The term “cluster” relates such to a discrete grouping of proteins, and additionally of other compounds.

For “Live cell-free engineered” or “cell-free manufactured” is to be understood that the microparticles and nanoparticles are synthetically obtained, free of any live cell acting as source of said particles. Thus, the proteins forming the cluster although of live prokaryotic or eukaryotic cells origin, they are not assembled inside any cell or in the presence of cells within the recipient where assembly is performed. Thus, the particles are free of any compounds that could be present in case the particles where formed in live prokaryotic or eukaryotic cells (cell debris, cytoplasmic components, and membrane components).

The term “assembling agent” relates to any compound and/or physical condition allowing certain molecules, such as proteins or a mixture of proteins and lipids, to configure discrete units or groupings of molecules (in a mode of tridimensional scaffolds).

Depending on the assembling agent or conditions and of the nature of the proteins, said groupings can be organized or unorganized groupings.

The term “release of an amount of assembled self-contained proteins lower than 50% by weight in relation to the total weight of assembled self-contained protein”, means that the microparticle or nanoparticle is indeed a delivery system with a slow release profile, and the cluster configured by assembled proteins loses less than 50% w/w of the self-contained proteins over a period of time and at physiological conditions. Physiological conditions include a temperature from 34.5° C. to 42° C. (being normal from 36.5° C. to 37.5° C. (the said physiological temperature) and pH around 7 (6.5-7.8). This release is also to be understood as a mode of delivering said proteins in a particular media. The media can be, in particular, a tissue from a living organism in such a way that the particle is finally decomposed, in particular in a sustained way. Skilled person in the art will know the different modes of determining amounts of released protein and amounts of proteins retained in the cluster. A more particular mode is disclosed in Examples of this description.

The term “insoluble pellet” means that when manufactured in aqueous media and at a temperature from 4° C. to 30° C. (or at room temperature from 18° C. to 30° C.), the nano- or microparticles precipitate or they sediment at the bottom of the recipient where they are formed. Thus, the nano- or microparticles are in form of a “precipitated pellet” in the aqueous media where they were manufactured (at 4° C.-30° C.) and that is visible among a predetermined period of time allowing sedimentation, or by means of mild centrifugation, for example at 15.000 g. Mild centrifugation includes the conditions known for the skilled man in the art, said conditions allowing separation of the particles at the bottom of the recipient but preserving the functional structure of the same (i.e. not damaging or disrupting the particles and/or proteins conforming them).

The term “mechanically stable”, which means that the cluster of self-contained proteins remains structured, that is, they are maintained assembled forming the cluster, when submitted to particular sonication conditions. For “remain structured” is to be understood that the assembled self-contained proteins that form part of the cluster do not lose their tridimensional configuration, so that self-contained proteins maintain the form of a cluster (aggrupation, aggregation); that resembling natural inclusion bodies.

The term “denatured form” or “denatured proteins” means that the proteins have lost the at least the quaternary structure and tertiary structure, which is present in their native state, by application of some external stress or compound such as a strong acid or base, a concentrated inorganic salt, an organic solvent (e.g., alcohol or chloroform), radiation or heat.

The term “functional protein” when used in this description, relates to the widely accepted meaning of a protein that maintains, works or provides its purposed activity. For example if a protein has the capability of fluorescence emission, the protein is functional if it can emit such fluorescence, which is the purpose for which it was used or designed. If on the other hand usual function of the protein involves inhibition of growth factors, the functional protein will maintain such property.

As used herein, “targeting molecule” refers to a molecule having specificity for a particular cell, tissue, or organ. Preferred examples of targeting molecules include but are not limited to antibodies, growth factors, and polysaccharides.

The particle is a nanoparticle or a microparticle. The term “nanoparticle” as used herein, refers to a particle with at least two dimensions at the nanoscale, particularly with all three dimensions at the nanoscale. For analogy, the term “microparticle” as used herein, refers to a particle with at least two dimensions at the microscale, particularly with all three dimensions at the microscale. In a particular embodiment, the particle is from 50 nm to 50 μm. In particular, from 60 nm to 10 μm. More in particular, from 60 nm to 5 μm. Even more in particular, from 500 nm to 5 μm. In certain embodiments is from 60 nm to 1 μm, even more in particular from 60 nm to 900 nm. Other more particular sizes are from 60 nm to 200 nm. Protein particles of the invention and produced according to the methods disclosed therein include a distribution of microparticles and of nanoparticles. Thus, the term “protein nano- or microparticle” or the term protein-lipid “nano- or microparticle”, relates to a mixture or distribution of particles of different sizes including nanoparticles, microparticles or combinations of nano and microparticles.

As regards the shape of the nanoparticles or microparticles described herein, there are included spheres, polyhedral and rod-shape. Particularly, when the nanoparticle or microparticle is substantially rod-shaped with a substantially circular cross-section, such as a nanowire or a nanotube, microwire or microtube, the “nanoparticle” or “microparticle” refers to a particle with at least two dimensions at the nanoscale or microscale, these two dimensions being the cross-section of the nanoparticle or the microparticle.

In a particular embodiment of the first aspect, optionally in combination with any of the embodiments provided above or below, the particle is spherical or pseudospherical.

As used herein, the term “size” refers to a characteristic physical dimension. For example, in the case of a nanoparticle/microparticle that is substantially spherical, the size of the nanoparticle/microparticle corresponds to the diameter of the nanoparticle/microparticle. When referring to a set of nanoparticles/microparticles as being of a particular size, it is contemplated that the set can have a distribution of sizes around the specified size. Thus, as used herein, a size of a set of nanoparticles/microparticles can refer to a mode of a distribution of sizes, such as a peak size of the distribution of sizes. In addition, when not perfectly spherical (pseudospherical), the diameter is the equivalent diameter of the spherical body including the object. This diameter is generally referred as the “hydrodynamic diameter”, which measurements can be performed using a Wyatt Möbius coupled with an Atlas cell pressurization system. Transmission Electron Microscopy (TEM) images do also give information regarding diameters.

As used herein, the indefinite articles “a” and “an” are synonymous with “at least one” or “one or more.” Unless indicated otherwise, definite articles used herein, such as “the” also include the plural of the noun.

The expression “therapeutically effective amount” as used herein, refers to the amount of a compound that, when administered, is sufficient to prevent development of, or alleviate to some extent, one or more of the symptoms of the disease which is addressed. The particular dose of compound administered according to this invention will of course be determined by the particular circumstances surrounding the case, including the compound administered, the route of administration, the particular condition being treated, and the similar considerations.

The term “cosmetic effective amounts” is defined as any amount sufficient to significantly improving the cosmetic appearance of the skin without substantial irritation, but low enough to avoid serious side effects (at a reasonable benefit/risk ratio), within the scope of sound medical judgement. The safe and effective amount of the particles of the invention will vary with the age and physical condition of the consumer, the condition of the skin, the duration of the treatment, the nature of any concurrent treatment, the specific combination of active ingredients employed, the particular cosmetically acceptable carrier utilized, and like factors in the knowledge and expertise of any attending physician.

The expression “pharmaceutically acceptable excipients or carriers” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio.

The term “cosmetically acceptable” or “dermatological acceptable” which is herein used interchangeably refers to that excipients or carriers suitable for use in contact with human skin without undue toxicity, incompatibility, instability, allergic response, among others.

As above indicated, present invention relates to protein nano- or microparticles, which are live cell free manufactured in a recipient, comprising clusters of one or more types of assembled self-contained proteins, and one or more salts of divalent cations, being the ratio of moles of salt of divalent cation:moles of self-contained protein comprised from 4:1 to 1000:1, wherein the nano- or micro particles:
have a size, measured as hydrodynamic diameter, from 50 nanometers (nm) to 50 micrometers (μm);
are mechanically stable, which means that the cluster of self-contained proteins remains structured, which means that does not lose the tridimensional configuration, when submitted at sonication conditions including 5 rounds of 40 seconds; 0.5 of pulse on; 0.5 of pulse off and a wave width of 10% in a high intensity sonicator Branson sonifier 450, with 3 mm-diameter titanium probe;
are in the form of a precipitated pellet in aqueous media, when centrifuged at 15.000 g at a temperature from 4° C. to 30° C.; and
they release an amount of assembled self-contained proteins lower than 50% by weight in relation to the total weight of assembled self-contained protein, within 24 hours and when resuspended in an aqueous media at physiological temperature.

In some particular embodiments, the particles release at least 50% by weight of the assembled self-contained proteins within at least 15 days while resuspended in aqueous media at physiological temperature.

In a particular embodiment, protein nano- or microparticles comprise a cluster made of assembled self-contained proteins of one or more types, which proteins are so assembled or aggregated due to the presence of salts of divalent cations within the scaffold (or cluster). Thus, in a particular embodiment, the protein nano- and/or microparticle according to the first aspect further comprise one or more salts of divalent cations.

Salts of divalent cations include single and multiple salts (i.e. double salts), and combinations thereof. In a more particular embodiment the divalent cations of the salts are selected from the group consisting of Be²⁺, Mg²⁺, Ca²⁺, Sr²⁺, Ba²⁺, Ra²⁺Zn²⁺, Cu²⁺ and Ni²⁺, and combinations thereof. In a particular embodiment, divalent cations of the salts are alkaline-earth cations. The salts are, in particular embodiment inorganic salts of divalent cations, more in particular of Be²⁺, Mg²⁺, Ca²⁺, Sr²⁺, Ba²⁺, Ra²⁺, Zn²⁺, Cu²⁺ and Ni²⁺, and combinations thereof. Particular salts include CaCl₂, ZnCl₂, NiCl₂, and combinations thereof. More in particular, the salt is ZnCl₂.

In another particular embodiment of the first aspect, optionally in combination with any embodiment above or below, the assembled self-contained proteins comprise one or more amino acids that due to the presence of charge at physiological pH and/or of the presence of aromatic or heteroaromatic structures can coordinate with the divalent cations. In another particular embodiment of the first aspect, optionally in combination with any embodiment above or below, the assembled self-contained proteins comprise one or more histidine residues. Thus, they are histidine-containing proteins.

In another particular embodiment, optionally in combination with any embodiment above or below, the cluster of protein molecules comprises His-tagged proteins. His tagged proteins are also known as histidine-rich proteins, which are proteins, usually recombinant proteins, comprising a polyhistidine-tag. His-tagged proteins according to present invention also include proteins with a number of histidines in their amino acid sequence selected from 3, 4, 5, 6, 7, 7, 9 and 10 histidines. The polyhistidine-tag is an amino acid motif in proteins that usually consists of at least six histidine (His) residues, often at the N- or C-terminus of the protein. Some proteins also comprise these at least six histidine residues in the middle of their amino acid sequence, such as in loop regions. This motif of histidine residues is also known as hexa histidine-tag, 6×His-tag, His6 tag and by the trademarked name His-tag (registered by EMD Biosciences). His-tagged proteins according to present invention also include natural proteins that comprise high amounts of histidine amino acid in their sequences.

In another particular embodiment of the first aspect, optionally in combination with any embodiment above or below, the assembled self-contained proteins comprises non-fibrous proteins or, in other words, the proteins configuring the cluster of assembled proteins are selected from the group consisting of membrane proteins, globular proteins, disordered proteins, and combinations thereof. In another particular embodiment, the proteins configuring the cluster of assembled proteins are selected from alkaline phosphatase, β-galactosidase, and combinations thereof.

In another particular embodiment of the first aspect, the protein nano- or microparticle comprises self-contained proteins that are therapeutic proteins, said therapeutic proteins optionally covalently linked and/or conjugated to one or more additional different therapeutic agent. In this particular embodiment, the same proteins configuring the cluster or scaffold of the nano- or microparticle are the therapeutics that can be delivered when the particle is sustainly solubilized at physiological conditions within a predetermined period of time, which time is function not only of the nature of the protein itself but also of the other compounds that can be accompanying the self-contained proteins configuring the cluster, such as other functional proteins, small drug molecules or therapeutic agents, or accompanying lipid structures, as a mode of non-limiting examples.

Particular therapeutic proteins, which mean that they are peptides (4 to 30 amino acids) or polypeptides (from 31 amino acids) with a proved therapeutic effect, include enzymes (such as alkaline phosphatase and β-galactosidase), hormones (i.e. insulin, growth hormones, etc.), hematopoietic growth factors (erythropoietin and derivatives, cell stimulating factors, such as granulocyte colony stimulating factors, etc.), interferons (Interferons-α, -β, -γ), blood factors (i.e. Factor VIII, Factor IX), thrombolytic agents (tissue plasminogen activator), antibodies (monoclonal and polyclonal antibodies), tumor necrosis factors, virus surface antigens, and hormones (insulin, glucagon, gonadotrophins, growth hormone), cell membrane receptors and corresponding secreted versions (i.e. klotho protein). Therapeutic proteins also encompass peptide aptamers and affimer molecules.

Thus, particles of the invention can be used as medicaments, in particular in diseases where the effective amounts of the therapeutic proteins are the active ingredients.

In a particular embodiment, the self-contained proteins are therapeutic proteins with a therapeutic effect selected from an anti-tumor effect, an anti-inflammatory effect, an antibiotic effect, an anti-fungal effect, an anti-viral effect, a growth factor effect, a cell growth inhibitor effect, an anti-platelet effect, an anti-thrombotic effect, a thrombolytic effect, and combinations thereof. In a more particular embodiment, the self-contained proteins are therapeutic proteins with an anti-tumor effect.

In yet another particular embodiment, optionally in combination with the embodiments above or below, the protein nano- or microparticle is a protein-lipid nano- or microparticle that comprises a cluster of assembled self-contained proteins and one or more types of lipids assembled with the self-contained proteins. In this particular embodiment the nano- or microparticle comprises a cluster comprising proteins and lipids that configure a tridimensional scaffold (i.e structure) with self-contained one or more proteins and with lipids associated to these proteins. Interacting with this cluster comprising lipids, other compounds are present in another particular embodiment, said compounds selected from functional proteins different from that of the self-contained ones and/or small drugs (therapeutic agents). These other compounds are disposed within interstitial spaces of the cluster of lipids and proteins and/or are adhered (adsorbed) to the cluster, by means of ionic interactions, van der Waals forces, or they are covalently linked.

Thus, in another more particular embodiment of the protein-lipid nano- or microparticle:
the self-contained proteins are denatured proteins and together with the assembled lipids configure a tridimensional scaffold; and
the particle further comprises one or more functional proteins disposed within the tridimensional scaffold and/or adhered (adsorbed) thereto.

In another particular embodiment of the protein-lipid nano- or microparticle the one or more functional proteins are therapeutic proteins, optionally covalently linked and/or conjugated to a one or more additional different therapeutic agent.

In a more particular embodiment, the one or more lipids are selected from the group consisting of fatty acids, glycerophospholipids, sterols, sphingolipids, and combinations thereof. In a more particular embodiment, the glycerophospholipids are selected from phosphatidylcoline, phosphatidylserine, phosphatidylethanolamine, phosphatidylinositol, and combinations thereof. The sterols are in particular selected from cholesterol, phytosterol, and combinations thereof. The sphingolipids are selected from sphingosine, ceramide, sphingomyelin, glucosyl cerebroside, and combinations thereof. In particular the lipids are those encompassed in the three known categories; phospholipids, glycolipids and cholesterol. The one or more lipids are, in a particular embodiment, the common lipids in the membranes of cells of living beings.

In another particular embodiment of the first aspect, the one or more functional proteins are therapeutic proteins, which mean that they are peptides (4 to 30 amino acids) or polypeptides (from 31 amino acids) with a proved therapeutic effect, as previously disclosed for the self-contained proteins. Examples of therapeutic proteins include, again, enzymes, hormones (i.e. insulin, growth hormones, etc.), hematopoietic growth factors (erythropoietin and derivatives, cell stimulating factors, such as granulocyte colony stimulating factors, etc.), interferons (Interferons-α, -β, -γ), blood factors (i.e. Factor VIII, Factor IX), thrombolytic agents (tissue plasminogen activator), antibodies (monoclonal and polyclonal antibodies), tumor necrosis factors, virus surface antigens, and hormones (insulin, glucagon, gonadotrophins, growth hormone), cell membrane receptors and corresponding secreted versions (i.e. klotho protein). Therapeutic proteins also encompass peptide aptamers and affimer molecules.

In another particular embodiment, the functional proteins are configured as nanoparticles of self-assembled proteins in the presence of divalent cations, more in particular histidine-containing proteins, in the presence of divalent cations selected from the group consisting of Be²⁺, Mg²⁺, Ca²⁺, Sr²⁺, Ba²⁺, Ra²⁺Zn²⁺, Cu²⁺ and Ni²⁺, and combinations thereof. This particular embodiment will be in protein microparticles, in particular, with a size from 1 μm to 50 μm.

In another particular embodiment of the first aspect, the one or more functional proteins disposed within the scaffold or cluster of self-assembled proteins or of proteins and lipids can additionally be covalently linked to this scaffold. In this particular case and when the functional protein is the active to be released, the covalent links are hydrolysable bonds. More in particular, they are hydrolysable at physiological conditions (i.e., ester bonds, amide bonds).

In another particular embodiment, these one or more functional proteins are proteins covalently linked and/or conjugated to a therapeutic agent, or to more than one therapeutic agent. This means that besides the functional protein has a therapeutic effect per se, other molecules with therapeutic effect are also combined (covalently linked or not with the functional protein). These therapeutic agents are, in a particular embodiment small molecules 900 Da). In the alternative, the therapeutic agent linked or conjugated to the functional protein is another protein or peptide. In yet another particular embodiment the therapeutic agent and the functional protein constitute a fusion protein and comprise two or more polypeptide fragments operatively linked and produced by recombinant technologies (i.e exogenous expression in expressing cells). In this later case when fusion proteins are used one or more of the fused polypeptides are functional proteins and have therapeutic effect. The therapeutic effect of the fusion protein is in a particular embodiment a polyvalent therapeutic effect, which means that each of the fused polypeptides exerts a different effect that can be complementary to each other. In another particular embodiment, all the fused proteins exert the same therapeutic effect (i.e. all fused parts have an anti-tumor effect).

In yet another particular embodiment, the therapeutic agent, embedded or adhered to the cluster of the nano-or-microparticle, linked and/or conjugated to the one or more functional proteins or to the self-contained proteins in the particles, are selected from the group consisting of an anti-tumor agent, an anti-inflammatory molecule, an antibiotic, an anti-fungal molecule, an anti-viral molecule, a growth factor, a cell growth inhibitor, an anti-platelet agent, an anti-thrombotic agent, a thrombolytic agent, and combinations thereof.

Non-limiting examples of therapeutic agents of different nature, embedded, adhered or linked in the microparticles or nanoparticles are listed below:

Non-limiting examples of antibiotics include β-lactam antibiotics (penicillin derivatives, monobactams, and carbapenems; polymyxins, such as colistin; rifamycins; lipiarmycins; quinolones; sulfonamides; macrolides; lincosamides; tetracylines; bactericidal aminoglycosides; cyclic lipopeptides, such as daptomycin; glycylcylines, such as tigecycline; oxazolidinones, such as linezolid; and lipiarmycins, such as fidaxomicin.

In a similar way, non-limiting examples of anti-fungal molecules, are amphotericin B, candicidin, filipin, hamycin, natamycin, nystatin and rimocidin; azole anti-fungals, such as imidazoles, e.g. bifonazole, butoconazole, clotrimazole, econazole, fenticonazole, isoconazole, miconazole, omoconazole, oxiconazole, sertaconazole, sulconazole and tioconazole; triazoles, e.g. albaconazole, fluconazole, isavuconazole, itraconazole, posaconazole, ravuconazole, terconazole and voriconazole; and thiazoles, e.g. abafungin; allylamines, such as amorolfin, butenafine, naftifine and terbinafine; echinocandins, such as anidulafungin, caspofungin and micafungin; benzoic acid; ciclopirox olamine; flucytosine; griseofulvin; tolnaftate and undecylenic acid.

Non-limiting examples of anti-viral molecules, include virus-assisted protein (VAP) anti-idiotypic antibodies; amantadine; rimantadine; pleconaril; acyclovir; zidovudine (AZT); lamivudine; integrase; fomivirsen; rifampicin; zanamivir and oseltamivir, and anti-parasitic molecules, such as mebendazole; pyrantel pamoate; thiabendazole; diethylcarbamazine; ivermectrin; niclosamide; praziquantel; albendazole; praziquantel; rifampin; amphotericin B; melarosprol; elfornithine; metronidazole; tinidazole and miltefosin.

A further example of therapeutic agent include growth factors, such as adenomedullin (AM), angiopoietin (Ang), autocrine motility factor, bone morphogenetic proteins (BMPs), brain-derived neutrophic factor (BDNF), epidermal growth factor (EGF), erythropoietin (EPO), fibroblast growth factor (FGF), glial cell line-derived neutrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), mystatin (GDF-8), nerve growth factor (NGF), platelet-derived growth factor (PDGF), thrombopoietin (TPO), transforming growth factor alpha (TGF-α), transforming growth factor beta (TGF-β), tumor necrosis factor alpha (TNF-α), vascular endothelial growth factor (VEGF), and placental growth factor (PIGF)

In yet a more particular embodiment, the anti-tumor agent is selected from one or more of the categorical group consisting of a chemotherapy agent, a cytotoxic agent (i.e either of polypeptide nature or a small drug), and antiangiogenic agent, a pro-apoptotic agent, an anti-metastatic agent or anti-proliferative agent (i.e an anti-mitotic agent), an immune stimulating agent (i.e promoting immune response towards tumors), a polypeptide encode by a tumor suppressor gene, a toxin, and combinations thereof.

Particular examples of anti-tumor agents, which are compounds that finally aim the fight of a cancer process in a patient include, but are not limited to, farnesyl transferase inhibitors; alkylating agents, such as nitrogen mustards, e.g. mechlorethamine, cyclophosphamide, melphalan, chlorambucil, ifosfamide and busulfan; nitrosoureas, e.g. N-nitroso-N-methylurea (MNU), carmustine (BCNU), lomustine (CCNU), semustine (MeCCNU), fotemustine and streptozotocin; tetrazines, e.g. dacarbazine, mitozolomide and temozolomide and aziridines, e.g. thiotepa, mytomycin, diaziquone (AZQ); and cisplatines, e.g. cisplatine, carboplatin and oxaplatin; antimetabolites, such as anti-folates, e.g. methotrexate and pemetrexed; fluropyrimidines, e.g. fluorouracil and capecitabine; deocynucleoside analogues, such as cytarabine, gemcitabine, decitabine, Vidaza, fludarabine, nelarabine, cladribine, clofarabine and pentostatine; and thiopurines, e.g. thiguanine and mercaptopurine; anti-microtubule agents, such as vinca alkaloids, e.g. vincristine, vinblastine, vinorelbine, vindesine and vinflunine; and taxanes, e.g. paclitaxel and docetaxel; and podophyllotxin; topoisomerase inhibitors, such as irinotecan, topotecan, captothecin, etoposide, doxorubicin, mitoxantrone, teniposide, novobiocine, merbarone and aclarubicin; cytotoxic antibiotics, such as antracyclines, e.g. doxorubicin, daumorubicin, epirubicin, idarubicin, pirarubicin, aclarubicin, mitoxantrone, actinomycin, bleomycin, plicamycin, and mitomycin. Other particular anti-tumor agents include antibodies, in particular monoclonal antibodies, such as trastuzumab, rituximab, alemtuzumab, Ibritumomab tiuxetan, bevacizumab, cetuximab, or panatimumab.

Other therapeutic agents include nucleic acid aptamers, peptide aptamers and affimers.

In yet another particular embodiment of the first aspect of the invention, the protein nano- or microparticle further comprises a targeting molecule linked to the cluster of assembled self-contained proteins. This targeting molecule has specificity for a particular cell, tissue, or organ to which the particles of the invention have to be delivered. Preferred examples of targeting molecules include but are not limited to antibodies, growth factors, and polysaccharides.

Present invention also encompasses a method for the synthesis of a protein nano- or microparticles as defined above, wherein the method comprises the following steps:

(a) mixing in a recipient one or more types of proteins in a polar solvent, more in particular a polar buffered solvent;
(b) submitting the mixture of step (a) to protein assembly conditions to obtain a protein-nano- or microparticle comprising a cluster of assembled self-contained proteins; and
(c) isolating the nano- or microparticle.

Thus, the invention encompasses a protein nano- or microparticle comprising a cluster of one or more types of assembled self-contained proteins, wherein the nano- or micro particle:
has a size, measured as hydrodynamic diameter, from 50 nanometers (nm) to 50 micrometers (μm);
is mechanically stable, which means that the cluster of self-contained proteins remains structured when submitted at sonication conditions including 5 rounds of 40 seconds; 0.5 of pulse on; 0.5 of pulse off and a wave width of 10% in a high intensity sonicator Branson sonifier 450, with 3 mm-diameter titanium probe;
is in the form of a precipitated pellet in aqueous media, when centrifuged at 15.000 g at a temperature from 4° C. to 30° C.; and
it releases an amount of assembled self-contained proteins lower than 50% by weight in relation to the total weight of assembled self-contained proteins within 24 hours and when resuspended in an aqueous media at physiological temperature;
and wherein the protein nano- or microparticle is obtainable by a method comprising the steps of:
   (a) mixing in a recipient one or more types of proteins in a polar solvent, more in particular a polar buffered solvent;
   (b) submitting the mixture of step (a) to protein assembly conditions to obtain a protein-nano- or microparticle comprising a cluster of assembled self-contained proteins; and
   (c) isolating the nano- or microparticle.

In a more particular embodiment of the method of the second aspect and of the nano- or microparticle obtainable by the same, in step (b) the protein assembly conditions comprise the addition of salts to the mixture of step (a) at a final salt concentration of salts in the mixture allowing precipitation of the one or more proteins, and/or applying a protein-denaturation temperature.

In another more particular embodiment of the method of the second aspect and of the nano- or microparticle obtainable by the same, it comprises the following steps:

(a) mixing in a recipient one or more types of proteins in a polar solvent;
(b) adding to the mixture of step (a) a solution of salts of divalent cations at a final salt concentration of salts in the mixture allowing precipitation of the one or more proteins;
(c) isolating the precipitated one or more proteins from the solvent in which they were precipitated, which are protein nano- or microparticles comprising a cluster of assembled self-contained proteins, and optionally resuspending them in a fresh buffered solvent.

Fresh buffer means new polar solvent, comprising a buffer system for the pH control, and the salt concentration to have the ionic force (isotonic) required by the proteins.

Thus, in a particular embodiment of the protein nano- or microparticle of the first aspect, said protein nano- or microparticle is obtainable by a method comprising the steps of:

(a) mixing in a recipient one or more types of proteins in a polar solvent;
(b) adding to the mixture of step (a) a solution of salts of divalent cations at a final salt concentration of salts in the mixture allowing precipitation of the one or more proteins;
(c) isolating the precipitated one or more proteins from the solvent in which they were precipitated, which are protein nano- or microparticles comprising a cluster of assembled self-contained proteins, and optionally resuspending them in a fresh buffered solvent.

In a particular embodiment, step (c) of isolating is carried out by means selected from the group consisting of centrifugation, filtering, drying, and combinations thereof, the skilled man will know. In another more particular embodiment isolation is performed by means of centrifugation.

In another particular embodiment of the method of the second aspect and of the nano- or microparticle obtainable by the same, when salts of divalent cations are used the final ratio of moles of salt of divalent cation:moles of protein is comprised from 4:1 to 1000:1. This proportion will depend on the protein that is going to be used, and the skilled man will know how to accommodate the amounts. For instance, the number of histidine residues or of other amino acid residues that can made complex linkages with the divalent cations will be a variable when adjusting the proportions, mainly requiring low amounts of divalent cations when the number of this complexing to the cation residues is higher in the protein molecule. Thus, it will vary depending on the amino acids in the protein molecule that can chelate (coordinated linkage) with the divalent cations. In a particular embodiment of the method of the second aspect when salts of divalent cations are used, the one or more proteins comprise one or more histidine residues.

In another more particular embodiment of the method of the second aspect and of the nano- or microparticle obtainable by the same, the final ratio of moles of salt of divalent cation:moles of protein is comprised from 5:1 to 500:1. More in particular from 10:1 to 500:1, even more in particular is from 20:1 to 200:1. Particular preferred ratios are selected from 5:1, 10:1, 20:1, 30:1, 40:1, 50:1, 60:1, 70:1, 100:1 and 150:1. The skilled person will understand that these ratios, as well as those of 4:1 to 1000:1 will be maintained in the obtained nano- or microparticle defined according to the first aspect.

Particular salts of divalent cations are the same as indicated for the first aspect of the invention.

Thus, in a particular embodiment the invention encompasses a protein nano- or microparticle, which is a cell-free manufactured nano- or microparticle, comprising a cluster of one or more types of assembled self-contained protein, and one or more salts of divalent cations, being the ratio of moles of salt of divalent cation:moles of self-contained protein comprised from 4:1 to 1000:1, wherein the nano- or microparticle:
has a size, measured as hydrodynamic diameter, from 50 nanometers (nm) to 50 micrometers (μm);
is mechanically stable, which means that the cluster of self-contained proteins remains structured when submitted at sonication conditions including 5 rounds of 40 seconds; 0.5 of pulse on; 0.5 of pulse off and a wave width of 10% in a high intensity sonicator Branson sonifier 450, with 3 mm-diameter titanium probe;
is in the form of a precipitated pellet in aqueous media, when centrifuged at 15.000 g at a temperature from 4° C. to 30° C.; and
it releases an amount of assembled self-contained proteins lower than 50% by weight in relation to the total weight of assembled self-contained protein within 24 hours and when resuspended in an aqueous media at physiological temperature; and wherein the protein nano- or microparticle is obtainable by a method comprising the steps of:
(a) mixing in a recipient one or more types of proteins in a polar solvent;
(b) adding to the mixture of step (a) a solution of salts of divalent cations at a final ratio of moles of salt of divalent cation:moles of self-contained protein in the mixture comprised from 4:1 to 1000:1, to allow precipitation of the one or more proteins;
(c) isolating the precipitated one or more proteins from the solvent in which they were precipitated, which are protein nano- or microparticles comprising a cluster of assembled self-contained proteins, and optionally resuspending them in a fresh buffered solvent.

In another particular embodiment of the method, said method is for the synthesis of a protein-lipid nano- or microparticle as defined above and comprises the steps of:

(a) mixing in a recipient one or more types of proteins in a polar solvent;
(b) adding to the mixture of step (a) a solution of salts at a protein-denaturation salt concentration and/or submitting the mixture to protein-denaturation temperature allowing precipitation of the one or more proteins in denatured form (denatured proteins);
(c) isolating the precipitated one or more proteins from the solvent in which they were precipitated, and resuspending them in a fresh buffered solvent, said step of resuspending with a fresh solvent comprising:
(c.1) Mixing the precipitated protein with one or more lipids dissolved in an organic solvent and wherein the ratio of the amount of protein and lipids is from 0.8:1 to 1:0.8, more in particular 1:1;
(c.2) removing the organic solvent to obtain a dry film of denatured proteins and lipids on the surface of the recipient;
(c.3) suspending the dry film of step (c.2) with a buffered composition, said buffered composition optionally comprising one or more functional proteins in an aqueous media, while agitating the mixture under a controlled temperature from 4° C. to 8° C., to obtain a protein-lipid nano- or microparticle comprising a cluster of assembled self-contained proteins and one or more types of lipids assembled with the self-contained proteins, said cluster optionally comprising one or more functional proteins embedded and/or adsorbed within the cluster of assembled self-contained proteins and lipids;
(c.4) separate the protein-lipid nano- or microparticle from the remaining buffered composition.

Thus, in a particular embodiment of the protein nano- or microparticle of the first aspect, said protein-lipid nano- or microparticle is obtainable by a method comprising the steps of:

(a) mixing in a recipient one or more types of proteins in a polar solvent;
(b) adding to the mixture of step (a) a solution of salts at a protein-denaturation salt concentration while and/or submitting the mixture to protein-denaturation temperature allowing precipitation of the one or more proteins in denatured form (denatured proteins);
(c) isolating the precipitated one or more proteins from the solvent in which they were precipitated, and resuspending them in a fresh buffered solvent, said step of resuspending with a fresh solvent comprising:
(c.1) Mixing the precipitated protein with one or more lipids dissolved in an organic solvent and wherein the ratio of the amount of protein and lipids is from 0.8:1 to 1:0.8, more in particular 1:1;
(c.2) removing the organic solvent to obtain a dry film of denatured proteins and lipids on the surface of the recipient;
(c.3) suspending the dry film of step (c.2) with a buffered composition, said buffered composition optionally comprising one or more functional proteins in an aqueous media, while agitating the mixture under a controlled temperature from 4° C. to 8° C., to obtain a protein-lipid nano- or microparticle comprising a cluster of assembled self-contained proteins and one or more types of lipids assembled with the self-contained proteins, said cluster optionally comprising one or more functional proteins embedded and/or adsorbed within the cluster of assembled self-contained proteins and lipids;
(c.4) separate the protein-lipid nano- or microparticle from the remaining buffered composition.

In a particular embodiment of the methods, the polar solvent of step (a) is an aqueous buffered composition, more in particular an aqueous buffered composition at a pH from 6.8 to 7.5. More in particular is water with a buffer to adjust pH. Particular buffers include phosphate-buffered saline (containing disodium hydrogen phosphate, sodium chloride and, in some formulations, potassium chloride and potassium dihydrogen phosphate), Tris-glycine or Tris-HCl. Aqueous buffered compositions area also termed in this description as an “aqueous media”.

In a particular embodiment of the methods in which protein-denaturation temperature is applied allowing precipitation of the proteins, said temperature is comprised from 60° C. to 120° C. These temperatures allow obtaining denatured proteins. In another particular embodiment of the method, the protein-denaturation salt concentration is at least of 0.5 M in the final mixture of step (b). More in particular yet, step (b) of the method for the synthesis of protein-lipid nano- or microparticles is carried out at a temperature from 90° C. to 120° C. and at a salt concentration is at least of 0.5 M.

Particular salts in the method for synthesis of a protein-lipid nano- or microparticle, are selected from salts of monovalent cations, salts of divalent cations and combinations thereof. Particular salts of divalent cations are selected from the group previously indicated for the first aspect. More in particular salts are selected from the group consisting of NaCl, KCl, LiCl, MgCl₂, CaCl₂, ZnCl₂, and combinations thereof.

In another particular embodiment of the method for the synthesis of a protein-lipid nano- or microparticle, in step (c) of isolation and resuspension of the precipitated one or more proteins, step (c1) is performed with an organic solvent selected from the group consisting of ethanol, mixtures of chloroform and methanol, hexane, chloroform, methanol, and combinations thereof.

In another particular embodiment of the method for the synthesis of a protein-lipid nano- or microparticle, in step (c) of isolation and resuspension of the precipitated one or more proteins, step (c3) is performed with an aqueous buffered composition, more in particular an aqueous buffered composition at a pH from 6.8 to 7.5. This buffered composition comprises, in another more particular embodiment, one or more functional proteins in the aqueous media, in such a way that when the film of denatured proteins and lipids is resuspended with said buffer, the clusters are formed and they comprise the additional functional proteins embedded in the cluster or adhered (adsorbed) thereto

Particular recipients to carry out the methods are of a material selected from the group consisting of glass, polypropylene (HDPP or LDPP), and polyethylene. In another particular embodiment, the support is glass. Glass is preferably used when particular organic solvents that could dissolve some plastic types (polymer) are used.

The invention also relates to dry films comprising one or more lipids and one or more denatured proteins, which are self-assembled and configure a tridimensional cluster, net, said film disposed on a support.

For dry films is to be understood that the percentage of humidity in the film is from 0% to 30% of relative humidity, measured according to known standard methodologies.

In another particular embodiment of the film, the support is a flat surface of a material selected from the group consisting of glass, polypropylene (HDPP or LDPP), and polyethylene. In another particular embodiment, the support is glass. As indicated, glass is preferably used when particular organic solvents that could dissolve some plastic types are used.

The dry film comprising one or more lipids and one or more denatured proteins, which are self-assembled and configure a tridimensional cluster is obtainable by a method comprising:

(a) mixing in a recipient one or more types of proteins in a polar solvent;

(b) adding to the mixture of step (a) a solution of salts at a protein-denaturation salt concentration and/or submitting the mixture to protein-denaturation temperature allowing precipitation of the one or more proteins in denatured form (denatured proteins);

(c) isolating the precipitated one or more proteins from the solvent in which they were precipitated, and mixing the precipitated protein with one or more lipids dissolved in an organic solvent and wherein the ratio of the amount of protein and lipids is from 0.8:1 to 1:0.8, more in particular 1:1; and

(d) removing the organic solvent to obtain a dry film of denatured proteins and lipids on the surface of the recipient.

This film of the invention can be further resuspended with a buffered composition optionally comprising one or more desired functional proteins, as indicated above, to obtain the protein-lipid nano- or micro particles comprising the scaffold (or clusters) of assembled self-contained proteins and one or more types of lipids assembled with the self-contained proteins, said cluster optionally comprising one or more functional proteins embedded and/or adsorbed within the cluster of assembled self-contained proteins and lipids. Thus, resuspended film allow obtaining particles comprising the cluster with one or more lipids and one or more denatured proteins, which are self-assembled and configure a tridimensional net; and the one or more functional proteins disposed within the scaffold or adhered thereto.

Particular organic solvents are as disclosed above for the method of obtaining the nano- or microparticles.

The protein nano- or microparticle of the invention is for use as a medicament as an additional aspect.

In a particular embodiment, the particles are for use in the treatment of a disease selected from the group consisting of cancer, an immune disease, neurodegenerative disease, and combinations thereof. This embodiment can also be formulated as the use of the protein nano- or microparticle as defined above for the manufacture of a medicament for the treatment or prevention of a disease selected from the group consisting of cancer, an immune disease, neurodegenerative, and combinations thereof. The present invention also relates to a method for the treatment or prevention of a disease selected from the group consisting of cancer, an immune disease, neurodegenerative, and combinations thereof, comprising administering a pharmaceutically effective amount of the protein nano- or microparticle as defined above, together with pharmaceutically acceptable excipients or carriers, in a subject in need thereof, including a human. Particular cancers include colorectal cancer.

As indicated, one aspect of the invention is a pharmaceutical composition comprising a therapeutically effective amount of the protein nano- or microparticle disclosed above together with pharmaceutically acceptable excipients or carriers.

In particular embodiments, the pharmaceutical composition of the invention can be formulated in several forms that include, but are not limited to, solutions, tablets, capsules, granules, suspensions, dispersions, creams, ointments, powders, lozenge, chewable candy, candy bar, concentrate, drops, elixir, emulsion, film, gel, granule, chewing gum, jelly, oil, paste, pastille, pellet, soap, sponge, suppository, syrup, chewable gelatin form, or chewable tablet.

Examples of suitable pharmaceutically acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

The relative amounts of the protein nano- or microparticle, the pharmaceutically acceptable excipients, and/or any additional ingredients in a pharmaceutical composition of the invention will vary, depending upon the identity, size, and/or condition of the subject treated and further depending upon the route by which the composition is to be administered.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Excipients such as coloring agents, coating agents, sweetening, and flavoring agents can be present in the composition, according to the judgment of the formulator.

In a particular embodiment of the pharmaceutical composition, it is for subcutaneous administration.

The invention also relates to cosmetic compositions comprising a cosmetic effective amount of the protein nano- or microparticles disclosed above together with one or more appropriate cosmetically acceptable excipients or carriers. Particular cosmetic compositions comprise protein nano- or microparticles comprising clusters of assembled self-contained proteins and optionally other functional proteins embedded or adhered to said cluster, one or more of any of these proteins, either the assembled self-contained or the functional embedded or adhered, being selected from proteins commonly used in cosmetics and selected from collagen of any type, growth factors, elastin, fibrin, among others. These proteins and so the cosmetic compositions comprising them can be used in particular for skin care, in particular for ameliorating at least one of the following symptoms: roughness, flakiness, dehydration, tightness, chapping, and lack of elasticity.

Other more particular cosmetic compositions comprise additional cosmetic active ingredients adhered or embedded in the nano- or microparticles. Non-limiting examples of cosmetic actives include plant extracts, plant cell lysates, anti-wrinkle compounds, hydrating compounds, whitening compounds, cicatrisation compounds, anti-cellulite compounds, skin-tightening compounds, antioxidant compounds, etc. These entire compounds can be of different nature, even of peptide nature or isolated amino acids. They can also be derived from plant compounds, such as phytosterols, polyphenols, terpenes, etc., or of lipid nature, such as fatty acids, or even they can derive from nucleic acids. Also compounds of polysaccharide structure can be used (i.e. hyaluronic acid compounds), or carboxylic acids. Therefore, all those actives providing a cosmetic effect can be used in the cosmetic compositions of the invention.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention.

The following examples are provided by way of illustration, and they are not intended to be limiting of the present invention. Furthermore, the present invention covers all possible combinations of particular and preferred embodiments described herein.

### EXAMPLES

### Example 1. Method for Preparing Protein Microparticles and Nanoparticles (Synthetic IBs), Using as Pattern the Fusion Protein T22-GFP-H6

All process is to be carried out under sterility conditions.

Materials:

Sterile double distilled water. Sterile NaCl solution. Filtered PBS buffer 1× and concentrated and sterile solution of protein nanoparticles (NPs). Solution of lipids (egg phosphatidyl choline (Sigma Aldrich) or lipids found in natural cell membranes) in chloroform:methanol (2:1) or organic solvents that homogeneously dilute the lipids used

1—Two solutions (solution A and B) of equal concentration of fusion protein T22-GFP-H6 (SEQ ID NO: 1) in double distilled water were prepared. (A concentration of 1 mg/mL already used for in vitro tests is suggested). Solution A contained NaCl (0.5-2 M) and solution B did not contain salt.

SEQ ID NO: 1 corresponds to the following sequence, from N to C-terminal:

M R R W C Y R K C Y K G Y C Y R K C R G G S
S R S S S K G E E L F T G V V P I L V E L D
G D V N G H K F S V S G E G E G D A T Y G K
L T L K F I C T T G K L P V P W P T L V T T
L T Y G V Q C F S R Y P D H M K R H D F F K
S A M P E G Y V Q E R T I S F K D D G N Y K
T R A E V K F E G D T L V N R I E L K G I D
F K E D G N I L G H K L E Y N Y N S H N V Y
I T A D K Q K N G I K A N F K I R H N I E D
G S V Q L A D H Y Q Q N T P I G D G P V L L
P D N H Y L S T Q S A L S K D P N E K R D H
M V L L E F V T A A G I T H G M D E L Y K H
H H H H H

T22-GFP-H6 was in form of nanoparticles (12 nm) formed through assembly of GFP-containing monomers produced in E. coli in the presence of divalent cations, as disclosed by López-Laguna et al., “Assembly of histidine-rich protein materials controlled through divalent cations”, Acta Biomaterialia 2019, vol. no. 83, pp.: 257-264.

2—Solution A was heated for 20 to 40 min at 100° C.

3—Subsequently it was centrifuged at 15.000 g for 30 min at 4° C. The presence of pellets corroborated the precipitation of the protein. The well-labeled supernatant was separated and stored to evaluate the presence of non-precipitated protein residues. Subsequently, two more centrifugations were carried out in the same conditions as before (15.000 g for 30 min at 4° C.) and resuspending both times in the same initial volume of double distilled water.

4—Final pellet was separated from the supernatant (with a metal spatula) and placed in the bottom of a glass container (*). Subsequently, lipid (egg phosphatidylcholine (Sigma Aldrich)) was added. The volume of lipid contained the same amount (in milligrams) of protein in the glass tube. In addition, a volume of lipid as small as possible is recommended for this step. So that, the lipid in the organic solvent was highly concentrated (10 mg of lipid/ml resuspended in chloroform:methane 2.1)

5—Since the mixture of lipid and protein contained chloroform/methanol, these solvents were removed by means of N₂ flow. The mixture was dried until a film of protein and lipid was completely dried (residual humidity from 0% to 30%) at the bottom of the glass tube. This film (scaffold of self-assembled protein and lipid molecules that configures a porous tridimensional net) can be stored if desired to be used in the future.

6—Later this film of protein and lipid was resuspended with a volume of the functional (which means that it works or provides the purposed activity) fluorescent protein T22-GFP-H6 of SEQ ID NO: 1 (1 mg/ml of buffered solution) in non-denatured form. The sample was mixed to a visible homogeneity and then the entire volume was transferred back to a plastic container to continue the procedure.

7—The sample was kept at 2 to 8° C. (in cold chamber) throughout the night, to allow the interaction of the functional protein with the proteolipid film.

8—The next day the sample was centrifuged to 15000 g for 30 min at 4° C. to release the functional protein not bound. This centrifugation was repeated twice more. The supernatant was saved to evaluate how much protein did not bind to this film.

9—The last pellet contained the protein-lipid nano/microparticles according to the invention. This sample can be stored at −80° C. if not used.

(*) The need to operate in this stage in a glass container was due to the plastic dissolving in chloroform/methanol.

Therefore, preparation of synthetic IBs was performed by a method comprising the following steps:
(a) mixing one or more denatured proteins with one or more lipids previously dissolved in an organic solvent until an homogeneous mixture is obtained, wherein the ratio of the amount of protein and lipids is 1:1;
(b) removing the organic solvent to obtain a dry film of denatured proteins and lipids
(c) suspending the dry film of step (b) with composition comprising one or more functional proteins in an aqueous media while agitating the mixture under a controlled temperature from 4° C. to 8° C. to obtain the protein-lipid nano- or microprotein comprising the scaffold (or cluster) and the one or more functional proteins disposed within, embedded or adhered thereto in the surface.
(d) separate the protein-lipid nano- or microprotein from the remaining aqueous media.

With this method a scaffold or cluster of self-assembled protein molecules configuring a tridimensional net was obtained, said scaffold comprising also lipids as in the natural IBs. One functional protein (i.e. T22-GFP-H6) was added within a buffered solution and it was let enough time to allow it to be embedded or adsorbed (adhered) in the tridimensional net.

### Example 2. Characterization of Protein Microparticles and Nanoparticles (Synthetic IBs)

Size and Morphology:

Protein-lipid particles of Example 1 were analyzed by dynamic light scattering (DLS) and Field Emission Scanning Electron Microscopy (FESEM).

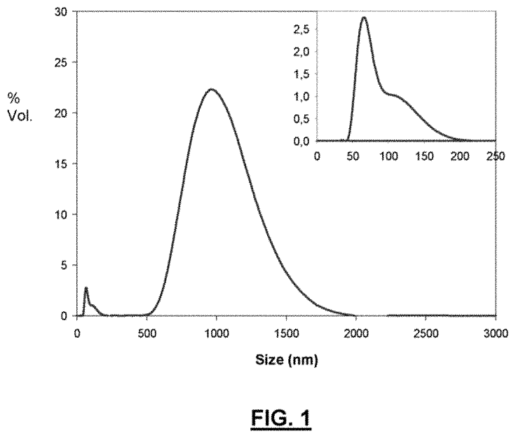

As can be seen in FIG. 1, which is a graphic of detected diameter (calculated by DLS) and the relative abundance of these diameters (% volume), the most of protein-lipid particles had a size around 1000 nm and they included particles from around 66 nm to 115 nm (small chart in FIG. 1).

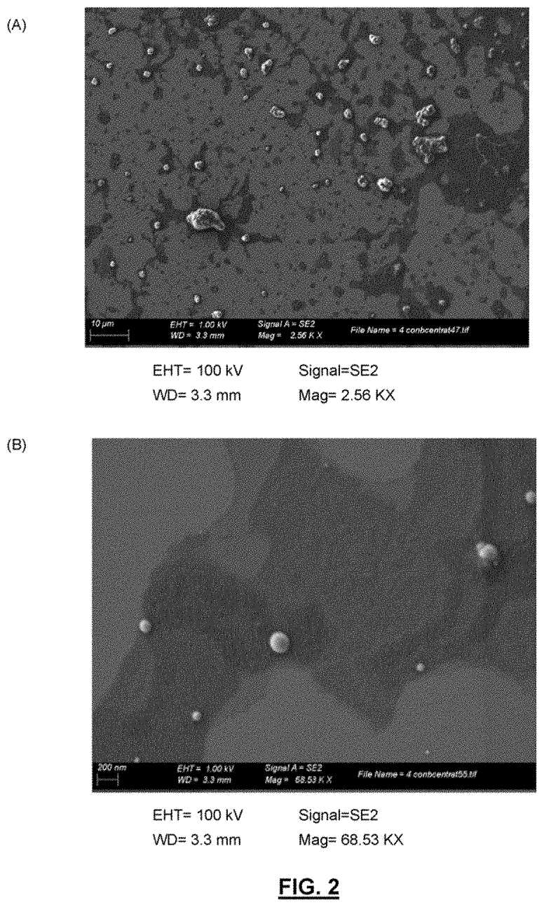

FIG. 2 shows FESEM images corroborating data of FIG. 1. FIG. 2 (A) shows synthetic IBs of big size and even some nanoparticles not previously detected. FIG. 2 (B) illustrates the nanoparticles of lower size also detected by DLS technology (FIG. 1). Images were obtained with the following adjusted device parameters: Electron high tension, (EHT)=1.00 kV; working distance (WD)=3.3 mm; Signal A=secondary electron (SE2); Mag=2.56 K X.

Thus, the particles of the invention have a size from 60 nm to 2000 nm, within the range of bacterial inclusion bodies.

### Example 3. Fluorescence Detection of T22-GFP-H6 in Protein-Lipid Microparticles and Nanoparticles of the Invention

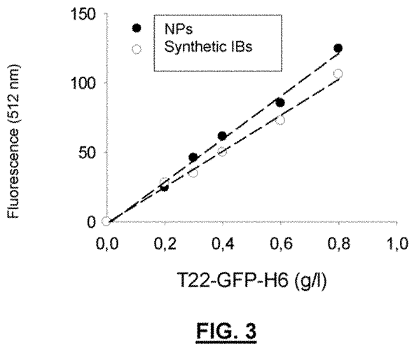

Fluorescence was evaluated with Cary Eclipse spectrofluorimeter (Agilent Technologies, Mulgrave, Australia), at a wave length of 512 nm] Data are depicted in FIG. 3, wherein for different concentrations of T22-GFP-H6 (g/l) relative intensity of fluorescence emitted at 512 nm is depicted for protein-lipid microparticles and nanoparticles of Example 1 (synthetic IBs; light circles). As control, fluorescence of the fusion protein in free form (not embedded in porous of lipid-protein scaffold) was also determined (NPs; dark circles). This free fusion protein was in form of nanoparticles (12 nm) formed through assembly of GFP-containing monomers produced in E. coli in the presence of divalent cations, as disclosed by López-Laguna et al., “Assembly of histidine-rich protein materials controlled through divalent cations”, Acta Biomaterialia 2019, vol. no. 83, pp.: 257-264.

Fluorescence was slightly lower in the particles in relation with the fluorescence of the protein nanoparticles originating them.

### Example 4. Protein Release from Protein-Lipid Microparticles and Nanoparticles of the Invention (Synthetic IBs)

For the analysis of soluble protein delivery of the particles obtained using the method of Example 1, artificial IBs in pellet format were resuspended in their corresponding buffers (without any additional protein) at a final concentration of 0.1 mg/mL and incubated without agitation at 37° C. (physiological temperature). Aliquots of 150 μl were taken at defined times (from 0 to 10 days) and centrifuged for 15 min, 4° C. at 15000 g. Soluble and insoluble fraction were separated and further processed by Tris-Glycine eXtended (TGX) Stain-Free electrophoretic gels and Western Blot. In all cases, isolated insoluble fractions were then resuspended in their respective buffer at the same final volume than their soluble counterpart for comparison.

Release of T22-GFP-H6 (SEQ ID NO: 1) in the cluster of protein-lipid microparticles and nanoparticles of Example 1 was determined.

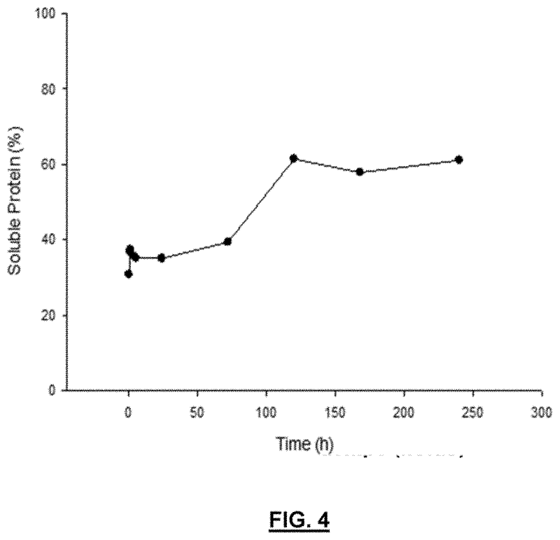

FIG. 4 shows the amount of released protein in % (i.e. release of functional fluorescent T22-GFP-H6) along time (Time in hours (h)).

A percentage by weight of 40% of protein in relation to the total protein was in the soluble fraction; meanwhile 60% remain in the form of a pellet.

### Example 5. Internalization of Protein-Lipid Microparticles and Nanoparticles of the Invention (Synthetic IBs) into Tumor Cells

The capability of IBs to penetrate tumor cells expressing was measured with in HeLa cell line (ATCC® CCL-2™, commercially available) expressing the cytokine receptor CXCR4 (CXCR4+ cells).

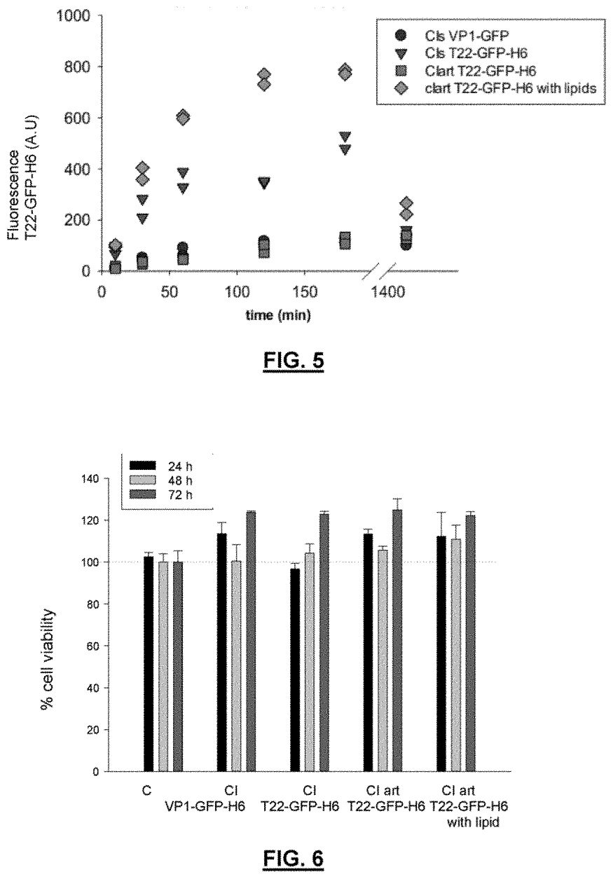

FIG. 5 is a graph with the detected fluorescence into the cells, determined by spectrofluorimeter (Agilent Technologies, Mulgrave, Australia), at a wave length of 512 nm. Different assays of internalization were performed using different entities for internalization:
CI VP1 GFP were IBs of bacterial origin and they are plotted as circles in FIG. 5.
CI T22-GFP-H6 were IBs of bacterial origin and they are plotted as triangles in FIG. 5
CI art (synthetic IBs) without lipid T22-GFP-H6, plotted as squares in FIG. 5 (manufactured as in Example 1 but without steps 4-5 and resuspending in step 6 the pellets of step 3 with a buffer containing functional protein of SEQ ID NO: 1)
CI art (synthetic IBs) T22-GFP-H6 was prepared according to Example 1, plotted as rhombus.

CI VP1 GFP and CI T22-GFP-H6 bacterial IBs were obtained following the protocol disclosed by Unzueta et al., “Engineering tumor cell-targeting in nanoscale amyloidal materials”, Nanotechnology-2017, vol. no. 28, pp.: 015102. Briefly, protein of SEQ ID NO:1 was cloned into pET22b (Novagen) vector. E. coli strain Origami B Novagen) was transformed with the expression vector. IB production was carried out in a shake flask in Lysogenum Brooth (LB) medium (37° C. and 250 rpm) until reach an optical density at 550 nm of 0.5. Gene overexpression was induced (isopropyl β-D-1-thiogalactopyranoside IPTG 1 mM) and the subsequent protein deposition as IBs. Samples of cultures producing SEQ ID NO: 1 were harvested by centrifugation (150000 g for 15 min at 4° C.). Pellets were resuspended in lysis buffer and with lysozyme. Mechanical disruption was carried out after enzymatic digestion (French press, five rounds at 1200 psi). Samples were then frozen (−80° C.) and after thawing IBs were washed while agitating. Several further steps of res-suspension and centrifugation, together with freeze and thaw cycles were performed until no viable bacteria were present.

As can be deduced from FIG. 5, protein-lipid microparticles and nanoparticles internalized efficiently into cells. So do the nanoparticles of T22-GFP-H6 but in a minor extent. Therefore, protein-lipid particles (synthetic IBs) do really act as natural bacterial IBs.

### Example 6. Cell Viability in Presence of Protein-Lipid Microparticles and Nanoparticles of the Invention (Synthetic IBs)

Toxicity of protein-lipid particles of the invention of Example 1 was determined at 24, 48 and 72 hours in CXCR4+ HeLa cells (ATCC® CCL-2™).

FIG. 6 is a graphic with the percentage of cell viability (% cell viability) of cells cultured with the presence of the following entities (as in Example 1):
CI VP1 GFP, IBs of bacterial origin.
CI T22-GFP-H6 IBs of bacterial origin.
CI art (synthetic IBs) without lipid T22-GFP-H6 (manufactured as in Example 1 but without steps 4-5 and resuspending in step 6 the pellets of step 3 with a buffer containing functional protein of SEQ ID NO: 1)
CI art (synthetic IBs) T22-GFP-H6 was prepared according to Example 1

The control (C) corresponds to the viability of the cells without the tested entities. It is used as 100% of cell viability.

The graphic shows that protein-lipid particles of Example 1 did not affect cell viability.

All these examples allow concluding that the particles of the invention, comprising a cluster of one or more types of assembled self-contained proteins configuring a tridimensional scaffold, are useful in the same way than natural bacterial inclusion bodies. They are stable, able to be internalized in tumor cells and they efficiently release any functional protein or even drug embedded within the cluster of assembled self-assembled protein molecules

Advantageously, drawbacks commonly associated (i.e. presence of bacterial components, non-homogeneity between batches, non-possibility of encapsulating proteins with post-translation modifications, and putative toxicity) with natural inclusion bodies are avoided with the protein nano- or microparticles according to the invention.

### Example 7. Method for Preparing Protein Microparticles and Nanoparticles (Synthetic IBs), Using as Pattern the Fusion Protein T22-GFP-H6 Using ZnCl₂ as Source of Divalent Cations

General procedure for this method of synthesis of protein nano- or microparticles of the invention is carried out according to the following detailed steps, herewith illustrated using protein T22-GFP-H6 (SEQ ID NO: 1):
1. Start from a purified protein sample in a specific storage buffer (generally sodium carbonate 166 mM with or without salt 333 mM) with a known concentration (generally above 2 mg/mL).
2. Start preparing a ZnCl₂ stock at a final concentration of 400 mM, final volume 50 mL in presence of distilled water.
3. After stock preparation, purified protein is diluted on its specific storage buffer till a final concentration of 2 mg/mL.
4. Separate the diluted protein in aliquots of 250 μL in eppendorfs.
5. Calculate the working proportion of 1:1 (ZnCl₂:Protein). At this step, the average or exact number of the histidine residues or of other amino acid residues that chelate with the divalent cation is needed. Below, there is exemplified the calculation considering the molecular weight of protein T22-GFP-H6 (SEQ ID NO: 1) and the six histidines comprised in the H6 tail segment of the fused protein

$$1 \frac{mg}{mL} \cdot \frac{1000 mL}{1 L} \cdot \frac{1 g}{1000 mg} \cdot \frac{1 mol}{30691.58 g} = 3.26 {10}^{- 5} \frac{mol}{L} = 0.0326 \frac{mmol}{L}$$

$$x 6 ( number of \frac{histidines}{protein} ) \to 0.0326 \frac{mmol}{L} \cdot 6 = 0.196 mM$$

6. This 1:1 proportion corresponded to 0.196 mM of ZnCl₂ and is the one to be used to precipitate the diluted protein (2 mg/mL).
7. Proportions from 5:1 to 500:1 showed a clear aggregation tendency. In this example, the following were the selected proportions for precipitation and corresponding ZnCl₂ concentration in mM:

| | ZnCl₂ |
|---|---|
| Proportions | concentration |
| (zinc | (mM) (1:1 = |
| chloride/protein) | 0.196) |
| Wilde type | 0 |
| 5:1 | 0.98 |
| 10:1 | 1.96 |
| 20:1 | 3.92 |
| 30:1 | 5.88 |
| 40:1 | 7.84 |
| 50:1 | 9.8 |
| 60:1 | 11.76 |
| 70:1 | 13.72 |
| 100:1 | 19.6 |
| 150:1 | 29.4 |

8. Specific volume of ZnCl₂ was added depending on the final mM concentration of each proportion, the final volume (250 μL) and stock concentration 400 mM using the following equation:

$${V}_{i} = \frac{{V}_{f} \cdot {c}_{f}}{{c}_{i}} = \frac{250 μ L \cdot {ZnCl}_{2} ( mM )}{400 mM}$$

9. Mixture of the protein and the salt was vortexed after adding ZnCl₂.
10. Precipitation of the protein (assembly of self-contained proteins) was seen as a formation of a mucus.
11. Mixtures were let for precipitation by waiting for 10 min at room temperature.
12. All samples at 15.000 g at 4° C. for 15 min were centrifuged.
13. Soluble from insoluble fraction were separated, this insoluble sample (pellets) corresponding to the protein nano- or microparticles (artificial IBs).
14. Protein concentration in the supernatant was quantified by Bradford assay.
15. By using the wild type as control (soluble protein sample without ZnCl₂ addition), the amount of formed precipitate in mg or in % was calculated.
   [i.e. If the quantification of the soluble fraction is nearly 0 of a specific proportion and the wild type concentration is nearly 2 mg/mL, then a precipitation of 100% was considered and the precipitate was practically 2 mg/mL (magnitude corresponding to a soluble format). In % by weight or in weight of protein, the values were obtained by multiplying 2 mg/mL of the precipitate to the used volume (250 μL-0.250 mL) obtaining 0.5 mg or the corresponding % in respect of the wild type control.]
16. After Bradford calculation, the % of precipitate in respect of the used proportion of ZnCl₂ was plotted in a graphic to extrapolate precipitation if needed in other proportions.
17. Additionally, protein precipitation could be seen by means of the fluorescence or non-fluorescent of the pellets after centrifugation.
18. Proportions 100 and 150 induced 100% of precipitation at this specific conditions.

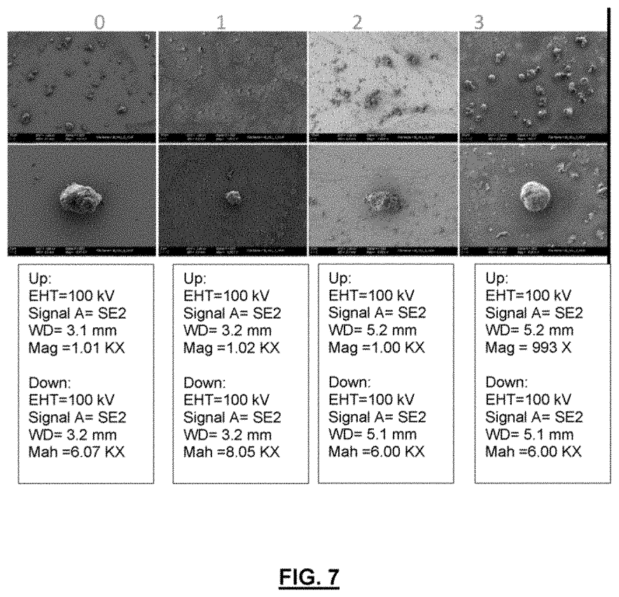

Protein nano and microparticles obtained following the above method can be seen in FESEM images of FIG. 7. This FIG. 7 includes images of several protein microparticles obtained by assembly of the fused protein T22-GFP-H6 with ZnCl₂ at different salt:protein molecular proportions. In images 1, 2 and 3 there are depicted the particles obtained using, respectively the molecular (or mol) proportions 40:1, 100:1 and 150:1 of salt:protein. Image 0 shows the image of a natural inclusion body produced directly in bacteria according to Unzueta et al, Nanotechnology 2017 (supra).

As can be seen in FIG. 7, with the method of the invention discrete protein particles resembling natural inclusions bodies were obtained. For this particular protein and salt of divalent cation, a proportion of moles of salt:moles of protein of 150:1 allow obtaining particle sizes similar to that of the natural bacterial inclusion body.

These data allow concluding that a method according to the invention is a good, reproducible and working method to obtain artificial inclusion bodies. In particular, that method comprising the steps of:

(a) mixing in a recipient one or more types of proteins in a polar solvent;
(b) adding to the mixture of step (a) a solution of salts of divalent cations at a final salt concentration of salts in the mixture allowing precipitation of the one or more proteins;
(c) isolating the precipitated one or more proteins from the solvent in which they were precipitated, which are protein nano- or microparticles comprising a cluster of assembled self-contained proteins, and optionally resuspending them in a fresh buffered solvent.

For the analysis of soluble protein delivery of the particles obtained using the method of divalent cations, artificial IBs in pellet format were resuspended in their corresponding buffers at a final concentration of 0.1 mg/mL and incubated without agitation at 37° C. (physiological temperature). Aliquots of 150 μl were taken at defined times (from 0 to 10 days) and centrifuged for 15 min, 4° C. at 15000 g. Soluble and insoluble fraction were separated and further processed by Tris-Glycine eXtended (TGX) Stain-Free electrophoretic gels and Western Blot. In all cases, isolated insoluble fractions were then resuspended in their respective buffer at the same final volume than their soluble counterpart for comparison.

At this assayed conditions (pH around 7), the solubility (release) of the particles at day 10 was of 30% w/w of the proteins in the soluble fraction and 70% w/w in the pellet (insoluble fraction).

Measured after 15 days when implanted subcutaneously, artificial IBs of this example were able to release 50% of the assembled self-contained proteins. This means that used as delivery system, these artificial IBs do really were effective and entered systemic route even when applied as an implant, although they are delivery systems with a slow release profile.

### Example 8. Manufacturing and Characterization of Protein Microparticles and Nanoparticles (Synthetic IBs or ArtIBs), Using Alkaline Phosphatese (AP), β-Galactosidase (β-Gal) and Fusion Proteins T22-GFP-H6 and T22-PE24-H6

Experimental Section

Fabrication of ArtIBs:

To produce multiple step ArtIBs (msArtIBs), microparticles and nanoparticles obtained in a particular method as indicated in Example 1, 1 mg of pure soluble protein was denatured and concomitantly precipitated by heating at 100° C. in NaCl₂ (500 mM), ZnCl₂ (26.4 mM) and MgCl₂ (18.4 mM) in distilled H₂O. The precipitate was centrifuged at 15,000 g for 15 min at 4° C., isolated from the soluble fraction and resuspended with 1 mg of phosphatidylcholine with chloroform/methanol 2:1 v/v in a final volume of 300 μL. The excess of organic solvent (OS) was removed by a continuous N₂ flow inducing the formation of a protein-lipid film phase that acted as scaffold. The scaffold was afterwards resuspended in 1 mg/mL of previous soluble protein diluted in PBS at 4° C. overnight. Finally, the sample was centrifuged, and soluble fraction discarded. The manufacturing of single step ArtIBs (ssArtIBs) was approached by diluting pure soluble protein in distilled H₂O at a final concentration of 2 mg/mL and final volume of 200 μL. Protein samples (0.196 mM) were subsequently mixed with ZnCl₂, at a 100:1 ratio of zinc to protein. After 10 min of incubation at room temperature samples were centrifuged at 15,000 g for 15 min and soluble fraction discarded to obtain the final product. Alternatively, zinc at a ratio 50:1 and calcium at a ratio 300:1 (in form of CaCl₂) were used for the in vivo experimental. β-galactosidase (β-Gal) [EC3.2.1.23] and alkaline phosphatase (AP) [EC3.1.3.1], both from Escherichia coli, were purchased from Sigma-Aldrich. T22-GFP-H6 (SEQ ID NO: 1) and T22-PE24-H6 (SEQ ID NO: 2) were produced as recombinant proteins and purified by single step chromatography.

SEQ ID NO 2 corresponds to the following sequence, from N to C-terminal:

MRRWCYRKCYKGYCYRKCRGGSSRSSRHRQPRGWEQ
LGGSPTGAEFLGDGGDVSFSTRGTQNVVTVERLLQ
AHAQLEERGYVFVGYHGTFLEAAQSIVFGGVAARS
QDLAAIWAGFYIAGDPALAYGYAQDQEPDAAGRIR
NGALLRVYVPASSLPGFYRTSLTLAAPEAAGEVER
LIGHPLPLALDAITGPEEEGGRLETILGWPLAERT
VVIPSAIPTDPRNVGGDLDPSSIPDKEQAISALPD
YASQPGKPPREDLKHHHHHHKDEL

Determination of Enzymatic Activity:

Between 3.7 and 21.3 ng of pure soluble β-Gal protein or β-Gal ArtIBs were mixed with 5 mM of ortho-nitrophenylgalactopiranoside (ONPG) in a final volume of 500 μL of PBS. The mixture was incubated for 15 min at 37° C., the reaction stopped by adding 200 μL of Na₂CO₃ (2.8 M) and the product amount determined by measuring absorbance at 420 nm (ε₄₂₀=4,530 M⁻¹·cm⁻¹) in a UV-Visible Spectrophotometer (Ultrospec 1000E, Pharmacia Biotech). On the other hand, between 3.9 and 92 ng of pure soluble AP protein or AP ArtIBs were mixed with 20 mM of para-nitrophenylphosphate (pNPP) in a final volume of 500 μL of PBS. The mixture was incubated for 15 min at 37° C., the reaction stopped by adding 200 μL of NaOH (1 M) and activity determined by measuring pNPP absorbance at 405 nm (ε₄₀₅=18,000 M⁻¹·cm⁻¹) in a UV-Visible Spectrophotometer (Ultrospec 1000E, Pharmacia Biotech).

Determination of Specific Fluorescence:

Pure soluble T22-GFP-H6 or ArtIB versions were diluted in PBS at concentrations ranging from 0.2 to 1 mg/mL. The excitation wavelength (λₑₓ) was set at 488 nm and the emission (λₑₘ) at 510 nm, meanwhile the excitation slit was set at 2.5 nm and the emission slit at 5 nm. Fluorescence was measured in a Cary Eclipse Fluorescence Spectrophotometer (Agilent Technologies) by using a quartz cell with a 10 mm path of light. The intrinsic fluorescence of each sample was then represented referred to protein concentration, defining the Specific Fluorescence Decay (SFD) mathematically represented as a slope. The % of SFD (% SFD) represent the relationship of the parameter with the SFD of soluble T22-GFP-H6 protein.

Size Distribution Analysis:

Volume Size Distribution of all nanostructures was determined at 633 nm and 25° C. in a Zetasizer Nano ZS (Malvern Instruments Limited) by using ZEN2112 3 mm quartz batch cuvettes. Protein samples dissolved in PBS from 0.2 to 1 mg/mL were measured in triplicate and mode size peak and polydispersion index (pdi±s.e.m.) obtained.

Electron Microscopy:

Ultrastructural morphometry (size and shape) of ArtIBs was characterized at nearly native state with field emission scanning electron microscopy (FESEM). Drops of 20 μl of each sample diluted at 0.3 mg/mL in their respective buffers were directly deposited on silicon wafers (Ted Pella Inc.) for 30 s and immediately observed without coating with a FESEM Zeiss Merlin (Zeiss) operating at 1 kV and equipped with a high resolution secondary electron detector. Representative images of a general fields and nanoparticle detail were captured at magnifications ranging between 5,500× and 8,500× and a working distance of 3.5 mm.

Attenuated Total Reflectance:

The most suitable concentration of ArtIBs was placed and dried with a continuous N₂ flow on spectroscopic crystal surfaces. Total Reflectance Spectroscopy was detected 15 times as spectra by using a scan rate of 50 cm^{-1/}min and a nominal resolution of 2 cm⁻¹ in a Tensor 27 Bruker spectrometer coupled to a Specac Golden Gate Attenuated Total Reflectance (ATR) accessory. All measurements were performed at 25° C., the absorbance obtained was corrected against the background and the PBS buffer signal was subtracted. Fourier deconvolution of the spectra and the second derivative allow the identification of the different band components. Fitting of the components to the original (not deconvolved) spectrum was essentially performed according to a described procedure [26]. Peak height, band width, and peak position of the components were allowed to vary one at a time in this order. A Gaussian shape was assumed.

Cell Culture:

CXCR4⁺ cervical cancer cell lines (HeLa ATCC® CCL-2™, commercially available) were used to study the performance of ArtIBs in vitro. Cells were routinely cultured in Eagle's Minimum Essential Medium (Gibco), supplemented with 10% fetal bovine serum (Gibco) and incubated in a humidified atmosphere at 37° C. and 5% of CO₂.

Protein Internalization:

HeLa CXCR4+ cells were cultured in 24-well plates in MEM Alpha 1× GlutaMAX™ medium (Gibco) supplemented with foetal bovine serum (FBS) at 37° C. in a 5% CO₂ humidified atmosphere until 70% of confluence was reached. The medium was then exchanged for serum free OptiPro medium (Gibco) before the addition of the protein. Protein uptake was determined at different times ranging from 10 min to 24 h at a final concentration of 2.5 μg. Cells were detached, and external hooked protein removed by Trypsin-EDTA (Gibco) at 1 mg/mL exposure for 15 min at 37° C. Intracellular protein fluorescence was detected by flow cytometry using a FACS-Canto system (Becton Dickinson) with an air-cooled argon ion laser (15 mW) exciting at 488 nm and a D detector (530/30 nm as bandpassfilter). In addition, the internalization specificity through CXCR4 receptor was tested by exposing cells to the CXCR4 antagonist AMD3100 (Sigma-Aldrich, (Saint Louis, Mo., USA) 1 h prior protein incubation at (protein/AMD3100) 1:10 ratio.

Cell Viability:

HeLa (ATCC-CCL-2, see above) cell line was cultured in opaque-walled 96-well plates at a final concentration of 6000 cells/well for 24 h. MEM Alpha GlutaMAX™ medium (Gibco) supplemented with foetal bovine serum (FBS) was used at 37° C. in a 5% CO₂ humidified atmosphere, until 70% of confluence was reached. ArtIBs were incubated at 1 μM for 96 h using MEM Alpha GlutaMAX™ medium (Gibco). Cell viability was measured by CellTiter-Glo®Luminescent Cell Viability Assay (Promega) in a Multilabel Plater Reader Victor3 (Perkin Elmer).

Soluble Protein Release from Artificial IBs:

ArtIBs were resuspended in 1 mL of PBS 1× reaching a final concentration of 1 mg/mL and incubated at 37° C. without agitation. 100 μL were taken from each sample at different times ranging from 0 to 7 days and centrifuged for 15 min at 15,000 g at 4° C. to isolate soluble and insoluble fractions. Soluble protein was then stain-free detected by TGX (TGX™ FastCast™ Acrylamide Kit) and subsequently quantified by ImageLab software to determine the % of released protein.

Results

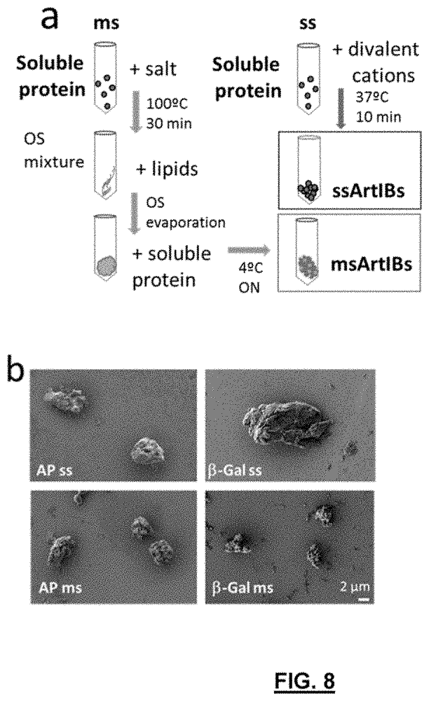

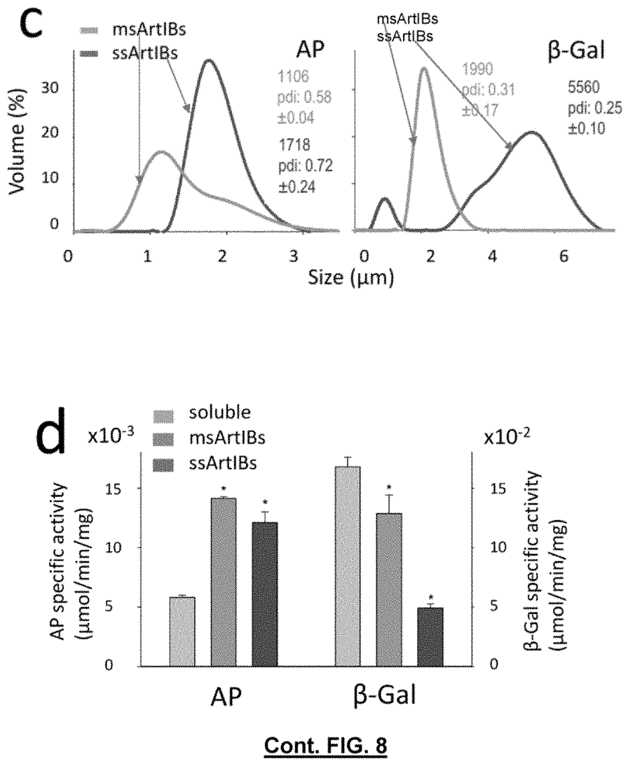

To explore the fabrication of synthetic IBs, common laboratory enzymes that form functional IBs during recombinant production, namely β-galactosidase (β-Gal) and alkaline phosphatase (AP), were selected as models for two alternative approaches to ArtIB fabrication. In one (FIG. 8 (A)), soluble protein was salted out plus thermally aggregated to generate amyloidal networks, further used as IB-like seeds to recruit and entrap homologous soluble protein versions. This multiple step procedure (ms), was aimed to imitate the dual, sponge-like networks in natural IBs. Lipids, common component of bacterial IBs were also incorporated. In a simpler single-step (ss) approach, divalent cations (Zn, in form of ZnCl₂), involved in amyloid formation and generically, in protein-protein-contacts, were added to a protein solution (FIG. 8(A)). The application of these procedures resulted in mechanically stable, discrete and moderately disperse protein particles sizing around 1-2 μm (AP) and 2-6 μm (β-Gal) (FIGS. 8 (B) and (C)), whose surface rugosity and amorphous appearance remembered those of natural IBs (FIG. 8 (B)). Both enzymes, in this packaged form, were enzymatically active (FIG. 8 (D)). On the other hand, cross-β-sheet amyloid like structure (ALS) was detected by Attenuated Total Reflectance (ATR) in AP ArtIBs at proportions (31-37%, ss and ms respectively) matching those of IBs.

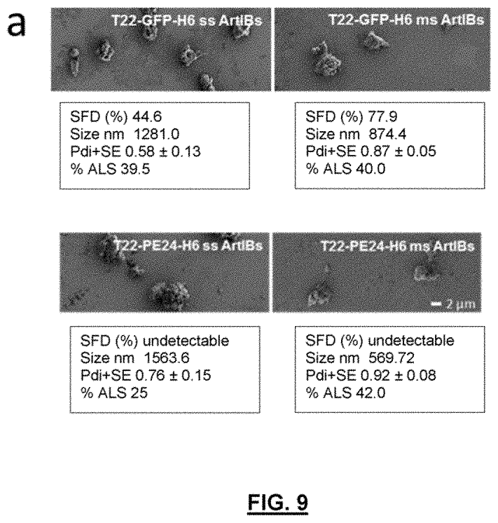

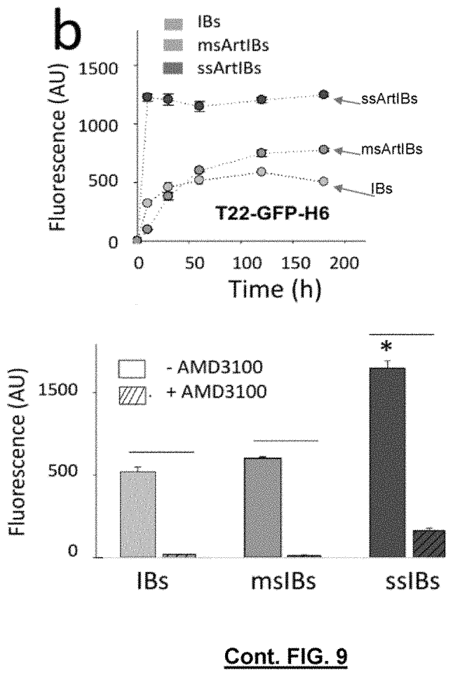

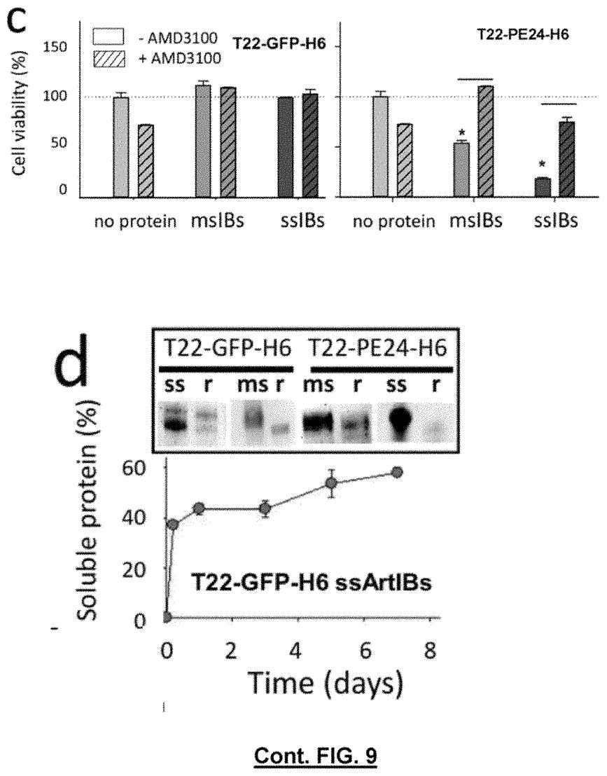

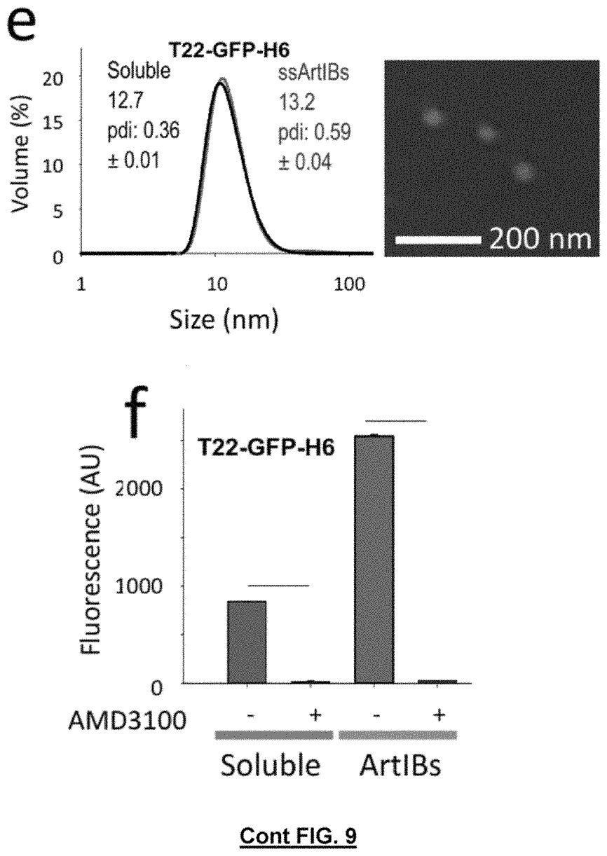

Further, new ArtIBs were constructed (FIG. 9 (A)) formed by the self-assembling modular proteins T22-GFP-H6 (SEQ ID NO: 1) and T22-PE24-H6 (SEQ ID NO: 2), that are targeted to the cell-surface cytokine receptor CXCR4 through the N-terminal tumor homing peptide T22. When exposed to cultured CXCR4⁺ Hela cells, T22-GFP-H6 ArtIBs internalized very efficiently as in the case of IB-based nanopills, by a CXCR4-dependent route inhibited by the CXCR4 antagonist AMD3100 (FIG. 9(B)). Cell viability was not affected by T22-GFP-H6 ArtIBs (FIG. 9 (C)), but it was instead dramatically compromised, in a CXCR4-dependent fashion, by the Pseudomonas aeruginosa exotoxin (PE24) contained in T22-PE24-H6 ArtIBs. As in the case of IBs, ArtIBs steadily released a fraction of the forming protein in soluble form when incubated in physiological buffer, at least for 7 days (FIG. 9 (D)). T22-GFP-H6 solubilized in vitro from ArtIBs was fluorescent (1039.83 AU/mg), assembled as 13 nm-nanoparticles indistinguishable in size from soluble T22-GFP-H6 (FIG. 9 (E)), and equally able to penetrate cultured HeLa cells in a CXCR4-dependent way (FIG. 9 (F)). This fact unveiled a potential of ArtIBs as chemically homogenous protein reservoirs for prolonged in vivo delivery of tumor-targeted, nanostructured protein drugs.

### Example 9. Subcutaneous Implant of Protein Microparticles and Nanoparticles (Synthetic IBs or Also Designated as ArtIBs) in an In Vivo Mouse Model of Colorectal Cancer

Experimental Section

In Vivo Release of Fluorescent Material by Subcutaneously Implanted ArtIBs and their Tumor Uptake:

Four-week-old female mice of the Swiss nude strain, in the 18-20 g body weight range (Charles River, L-Abreslle, France), maintained in Pathogen-free conditions, were used for the in vivo experiments. All experimental procedures were approved by the Hospital de Sant Pau Animal Ethics Committee and performed according to European Council directives. To generate the CXCR4⁺ SW1417 CRC cancer model, a 10 mg aliquot of SW1417-luci tumor tissue was obtained from donor animals and deposited in the anterior or posterior flank subcutis of the animals. When tumors reached approximately a 120-200 mm³ volume, animals were randomly allocated and implanted in the subcutis (SC) of the mouse lumbar region with a pellet of T22-GFP-H6 msArtIBs or T22-GFP-H6 ssArtIBs Zn²⁺ (both types manufactured as indicated in Example 8) in a preliminary study, at a single dose injection of 1 mg/mouse, suspended in a 150 μL PBS buffer, whereas in a second study, T22-GFP-H6 Zn²⁺ ArtIBs or of T22-GFP-H6 Ca²⁺ ArtIBs were SC implanted at the same dose. Control Buffer injection was used as a negative control. The ArtIBs injection point was selected to position it as far away as possible from the tumor in the same mouse, being located either in the anterior or posterior flanks.

After ArtIBs pellet injection, IVIS® Spectrum equipment (PerkinElmer Inc.) was used to monitor the GFP-emitted fluorescence by the SC implants in whole-body mouse by registering immediately (0 h) and at specific time points (3, 6 and 10 days) after the administration to determine the fluorescence remaining in the subcutaneous ArtIBs implants, as well as the fluorescent material that reached the remote tumor along time, in each mouse. Fluorescent signal was digitalized, displayed as pseudocolor overlay, and expressed as radiant efficiency. The fluorescence intensity (FLI) ratio was calculated dividing the signal from the IBs-treated mice by the FLI auto-fluorescent signal of Buffer-administered control mice either in the injection point or in the tumor.

In Vivo Antitumor Activity of SC Implanted ArtIBs:

The CXCR4+SW1417 CRC cancer model used to test antitumor activity was generated as described above. The expression of luciferase by cancer cells in this model allowed for the non-invasive follow-up of tumor growth along time. A week before the deposition of the tumor aliquot in the mouse subcutis, mice were randomly allocated to be SC administered in the mouse lumbar region with 1 mg/mouse dose of T22-GFP-H6 Ca²⁺ ArtIBs or T22-PE24-H6 Ca²⁺ ArtIBs suspended in a 150 μl of PBS buffer or Buffer-treated control mice. After IBs administration, mouse body weight was recorded, and bioluminescent image intensity (BLI) in the tumor, measured using the IVIS® Spectrum equipment (PerkinElmer Inc.), was digitalized and expressed as radiant efficiency. Tumor tissue, liver and kidney were formalin-fixed and paraffin-embedded for histology. To that aim, four-micrometer-thick sections were stained with hematoxylin and eosin (H&E), and analyzed for possible histological alterations by two independent observers. Representative images were taken using Cell\hat{ }B software (Olympus Soft Imaging v 3.3).

Statistical Analysis (Applicable to all Examples in this Description)

All analyses were performed with SPSS vs 11.0 (IBM) software. One-way ANOVA and t-tests were performed to assess differences in assays with a minimum n=3. The Holme-Sidak was applied for equal variance and Tukey or Mann Whitney U test for unequal variance (indicated in the figure legend). Two tail t-test was also used for individual comparisons. Data were presented as means±standard error of the mean (s.e.m). Differences between the protein samples were considered significant at p 0.05.

Results

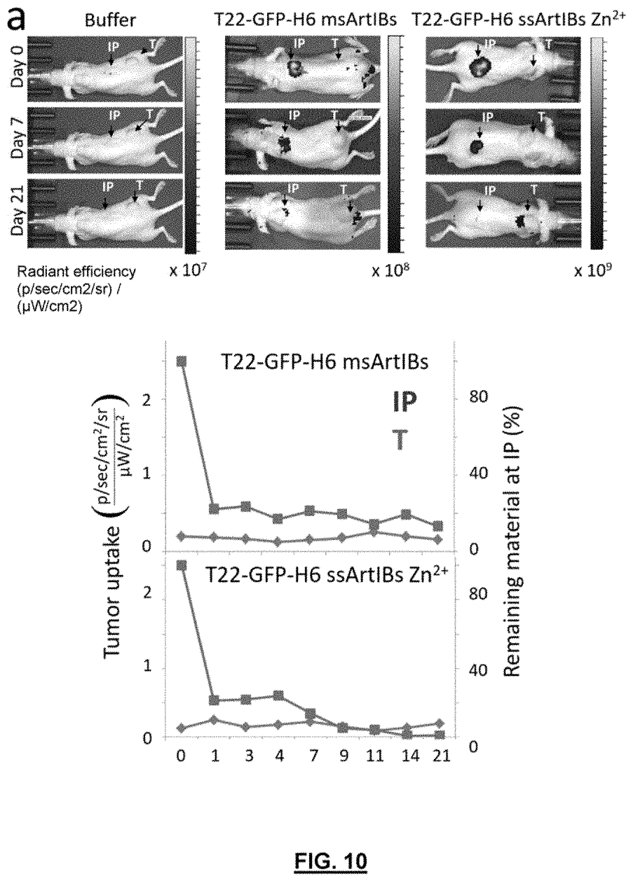

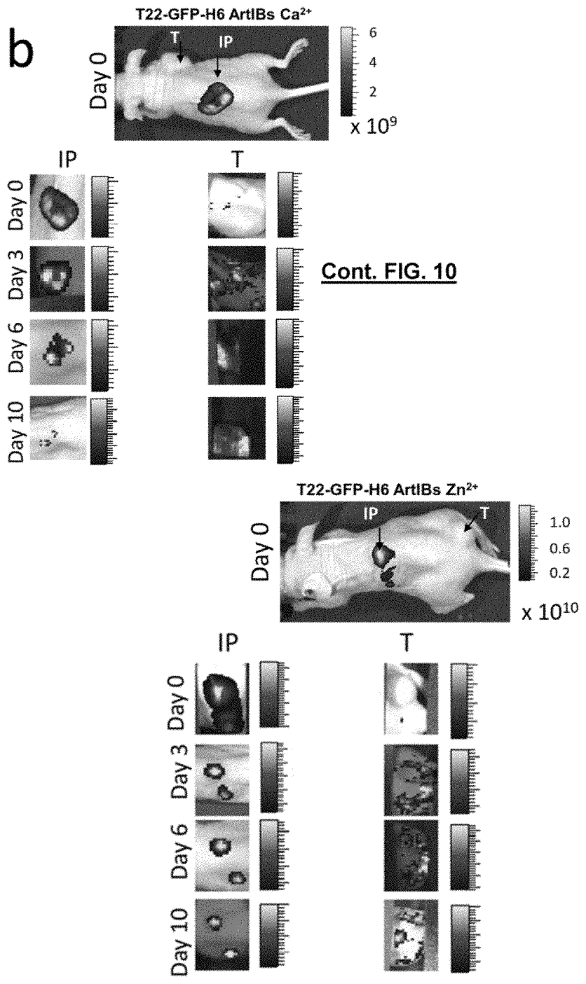

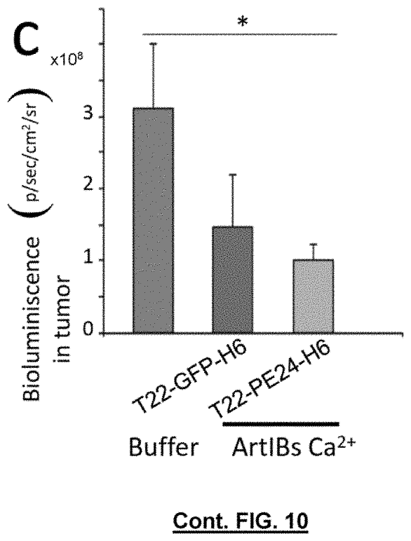

In the context of data obtained in Example 8, different categories of T22-GFP-H6 ArtIBs were implanted subcutaneously (SC) in a CXCR4⁺ colorectal cancer mouse model, releasing fluorescent material from the implantation point, followed by selective uptake by a remote CXCR4⁺ tumor, with specific kinetics for each ArtIB type. A preliminary screening of T22-GFP-H6 msArtIBs and T22-GFP-H6 ssArtIBs (Zn²⁺, at 100:1 ratio of zinc to protein) showed slow release and negligible or a small amount of material accumulated in the tumor by 21 days (FIG. 10 (A)). Lowering the proportion of Zn²⁺ (50:1) or using alternative cations to induce ssArtIBs formation improved release and tumor uptake. In particular, in this cancer model ssArtIBs formed by Ca⁺² were more efficient than ArtIBs (Zn⁺² 50:1) in maintaining a faster and progressive protein release from the SC injection site leading to a higher accumulation and longer residence time (starting at day 3 and at least until day 10) in the remote CXCR4⁺ tumor (FIG. 10(B)). Further, T22-PE24-H6 ArtIBs Ca²⁺, containing a CXCR4-targeted cytotoxic polypeptide (PE) and administered by the same route, induced a higher (p=0.083) inhibition of tumor growth (n=3, 1.0±0.2×10⁸) than T22-GFP-H6 Ca²⁺ ArtIBs (n=3, 1.5±0.7×10⁸), as compared to the control buffer-treated group (n=2, 2.6±1.0×10⁸), and as measured by bioluminescence emission (Average Radiance) at day 7 after ArtIB implantation (FIG. 10(C)). This occurred in absence of systemic toxicity (lack of histopathological alterations in H&E-stained liver and kidney at the end of the experiment, not shown). These observations fully confirmed both the secretion-like prolonged protein release and the precise cell targeting of functional materials through the blood stream, from a remote location.

In summary, protein microparticles and nanoparticles of the invention (abbreviated as ArtIBs or synthetic IBs) can be fabricated in vitro as a new type of biomimetic material, from pure protein and by simple physicochemical methods. These protein particles reproduce main IB properties that are relevant to potential uses in biomedicine, specially protein release. In particular, the simpler single step fabrication method allows engineering the strength of protein-protein interactions in the material by means of the stoichiometric control of metal or non-metal divalent cations. In contrast to other biocompatible materials developed for slow drug and protein drug release, ArtIBs are chemically homogeneous and show no distinction between carrier and cargo, thus acting as self-contained drug materials. ArtIBs of the invention might not only replace IBs as functional protein reservoirs and offer homogeneous materials for drug-oriented development, but they enable, in addition, to package glycosylated proteins of mammalian cell origin as IB-like materials. Since these proteins would be never produced in bacteria in functional forms, ArtIBs will then expand, as a universal platform, the catalogue of enzymes or protein drugs that could be formulated as pure micro-scale biocatalysts or as secretory protein granules.

Further aspects/embodiments of the present invention can be found in the following clauses:

Clause 1.—Protein nano- or microparticle comprising a cluster of one or more types of assembled self-contained protein, wherein the micro/nanoparticle:
has a size, measured as hydrodynamic diameter, from 50 nanometers (nm) to 50 micrometers (μm);
is mechanically stable, which means that the cluster of self-contained proteins remains structured when submitted at sonication conditions including 5 rounds of 40 seconds; 0.5 of pulse on; 0.5 of pulse off and a wave width of 10% in a high intensity sonicator Branson sonifier 450, with 3 mm-diameter titanium probe;
is in the form of a precipitated pellet in aqueous media, when centrifuged at 15.000 g at a temperature from 4° C. to 30° C.; and
it releases an amount of assembled self-contained proteins lower than 50% by weight in relation to the total weight of assembled self-contained protein within 24 hours and when resuspended in an aqueous media at physiological temperature.

Clause 2.—The protein nano- or microparticle according to clause 1, further comprising one or more salts of divalent cations.

Clause 3.—The protein nano- or microparticle according to clause 2, wherein the divalent cations are selected from the group consisting Be²⁺, Mg²⁺, Ca²⁺, Sr²⁺, Ba²⁺, Ra²⁺Zn²⁺, Cu²⁺, Ni²⁺, and combinations thereof.

Clause 4.—The protein nano- or microparticle according to any one of clauses 1-3, wherein the self-contained proteins are therapeutic proteins, said therapeutic proteins optionally covalently linked and/or conjugated to one or more additional different therapeutic agent.

Clause 5.—The protein nano- or microparticle according to any one of clauses 1-3, which is a protein-lipid nano- or microparticle that comprises a cluster of assembled self-contained proteins and one or more types of lipids assembled with the self-contained proteins.

Clause 6.—The protein-lipid nano- or microparticle according to clause 5, wherein:
the self-contained proteins are denatured proteins and together with the assembled lipids configure a tridimensional scaffold; and
the particle further comprises one or more functional proteins disposed within the tridimensional scaffold or adhered thereto.

Clause 7.—The protein-lipid nano- or microparticle according to any one of clauses 5-6, wherein the lipids are selected from the group consisting of fatty acids, glycerophospholipids, sterols, sphingolipids, and combinations thereof.

Clause 8.—The protein-lipid nano- or microparticle according to any ones of clauses 5-7, wherein the one or more functional proteins are therapeutic proteins, optionally covalently linked and/or conjugated to a one or more additional different therapeutic agent.

Clause 9.—Method for the synthesis of a protein nano- or microparticle as defined in any of clauses 1 to 8, wherein the method comprises the following steps:

(a) mixing in a recipient one or more types of proteins in a polar solvent;
(b) submitting the mixture of step (a) to protein assembly conditions to obtain a protein-nano- or microparticle comprising a cluster of assembled self-contained proteins; and
(c) isolating the nano- or microparticle.

Clause 10.—The method according to clause 9, wherein in step (b) the protein assembly conditions comprise the addition of salts to the mixture of step (a) at a final salt concentration of salts in the mixture allowing precipitation of the one or more proteins, and/or applying a protein-denaturation temperature.

Clause 11.—The method according to any one of clauses 9-10, wherein the method comprises the following steps:

(a) mixing in a recipient one or more types of proteins in a polar solvent;
(b) adding to the mixture of step (a) a solution of salts of divalent cations at a final salt concentration of salts in the mixture allowing precipitation of the one or more proteins;
(b) isolating the precipitated one or more proteins from the solvent in which they were precipitated, and optionally resuspending them in a fresh buffered solvent.

Clause 12.—The method according to any one of clauses 9-10, which is a method for the synthesis of a protein-lipid nano- or microparticle comprising the steps of:

(a) mixing in a recipient one or more types of proteins in a polar solvent;
(b) adding to the mixture of step (a) a solution of salts at a protein-denaturation salt concentration while and/or submitting the mixture to protein-denaturation temperature allowing precipitation of the one or more denatured proteins;
(c) isolating the precipitated one or more proteins from the solvent in which they were precipitated, and resuspending them in a fresh buffered solvent, said step of resuspending with a fresh solvent comprising:
(c.1) Mixing the precipitated protein with one or more lipids dissolved in an organic solvent and wherein the ratio of the amount of protein and lipids is from 0.8:1 to 1:0.8;
(c.2) removing the organic solvent to obtain a dry film of denatured proteins and lipids
(c.3) suspending the dry film of step (c.2) with a buffered composition, optionally comprising one or more functional proteins, while agitating the mixture under a controlled temperature from 4° C. to 8° C. to obtain a protein-lipid nano- or microparticle comprising a cluster of assembled self-contained proteins and one or more types of lipids assembled with the self-contained proteins, said cluster optionally comprising one or more functional proteins embedded and/or adsorbed within the cluster of assembled self-contained proteins and lipids;
(c.4) separate the protein-lipid nano- and/or microparticle from the remaining buffered composition.

Clause 13.—A protein nano- or microparticle as defined in any of clauses 1-8, for use as a medicament.

Clause 14.—The protein nano- or microparticle for use according to clause 13, which is for use in the treatment of a disease selected from the group consisting of cancer, an immune disease, neurodegenerative disease, and combinations thereof.

Clause 15.—The protein nano- or microparticle for use according to any of clauses 13-14, which is for use in the treatment of cancer.

Clause 16.—The protein nano- or microparticle for use according to clause 15, wherein the cancer is colorectal cancer.

Clause 17.—A pharmaceutical composition comprising a therapeutically effective amount of the protein a nano- or microparticle as defined any of clauses 1-8, together with pharmaceutically acceptable excipients or carriers.

Clause 18.—The pharmaceutical composition according to clause 17, which is for subcutaneous administration.

### CITATION LIST

Neubauer, et al., “Protein inclusion bodies in recombinant bacteria”, 237-292, Springer-2006.
Garcia-Fruitos, E. et al. “Aggregation as bacterial inclusion bodies does not imply inactivation of enzymes and fluorescent proteins”, Microbial cell factories 2005, vol. no. 4, 27, doi:10.1186/1475-2859-4-27.
Gonzalez-Montalban, N., et al., “Recombinant protein solubility—does more mean better?”, Nature biotechnology 2007, vol. no. 25, pp.: 718-720, doi:10.1038/nbt0707-718.
López-Laguna et al., “Assembly of histidine-rich protein materials controlled through divalent cations”, Acta Biomaterialia 2019, vol. no. 83, pp.: 257-264.
Unzueta et al., “Engineering tumor cell-targeting in nanoscale amyloidal materials”, Nanotechnology-2017, vol. no. 28, pp.: 015102

1. Protein nano- or microparticle, which is a cell-free manufactured protein nano- or microparticle, comprising a cluster of one or more types of assembled self-contained protein, and one or more salts of divalent cations, being the ratio of moles of salt of divalent catiommoles of self-contained protein comprised from 4:1 to 1000:1, wherein the micro/nanoparticle:
has a size, measured as hydrodynamic diameter, from 50 nanometers (nm) to 50 micrometers (μm);
is mechanically stable, which means that the cluster of self-contained proteins remains structured when submitted at sonication conditions including 5 rounds of 40 seconds; 0.5 of pulse on; 0.5 of pulse off and a wave width of 10% in a high intensity sonicator Branson sonifier 450, with 3 mm-diameter titanium probe;
is in the form of a precipitated pellet in aqueous media, when centrifuged at 15.000 g at a temperature from 4° C. to 30° C.; and
it releases an amount of assembled self-contained proteins lower than 50% by weight in relation to the total weight of assembled self-contained protein within 24 hours and when resuspended in an aqueous media at physiological temperature.

2. The protein nano- or microparticle according to claim 1, wherein the divalent cations are selected from the group consisting Be²⁺, Mg²⁺, Ca²⁺, Ba²⁺, Ra²⁺ Zn²⁺, Cu²⁺, Ni²⁺, and combinations thereof.

3. The protein nano- or microparticle according to claim 1, wherein the self-contained proteins are therapeutic proteins, said therapeutic proteins optionally covalently linked and/or conjugated to one or more additional different therapeutic agent.

4. The protein nano- or microparticle according to claim 1, which is a protein-lipid nano- or microparticle that comprises a cluster of assembled self-contained proteins and one or more types of lipids assembled with the self-contained proteins.

5. The protein-lipid nano- or microparticle according to claim 4, wherein:
the self-contained proteins are denatured proteins and together with the assembled lipids configure a tridimensional scaffold; and
the particle further comprises one or more functional proteins disposed within the tridimensional scaffold or adhered thereto.

6. The protein-lipid nano- or microparticle according to claim 4, wherein the lipids are selected from the group consisting of fatty acids, glycerophospholipids, sterols, sphingolipids; and combinations thereof.

7. The protein-lipid nano- or microparticle according to claim 4, wherein the one or more functional proteins are therapeutic proteins, optionally covalently linked and/or conjugated to a one or more additional different therapeutic agent.

8. Method for the synthesis of a protein nano- or microparticle as defined in claim 1, wherein the method comprises the following steps:
(a) mixing in a recipient one or more types of proteins in a polar solvent;
(b) submitting the mixture of step (a) to protein assembly conditions to obtain a protein-nano- or microparticle comprising a cluster of assembled self-contained proteins, said protein assembly conditions comprising adding to the mixture of step (a) a solution of salts of divalent cations at a final ratio of moles of salt of divalent cation:moles of protein comprised from 4:1 to 1000:1 and allowing precipitation of the one or more proteins, and/or applying a protein-denaturation temperature; and
(c) isolating the nano- or microparticle.

9. A method for the synthesis of a protein-lipid nano- or microparticle as defined in claim 4, comprising the steps of:
(a) mixing in a recipient one or more types of proteins in a polar solvent;
(b) adding to the mixture of step (a) a solution of salts at a protein-denaturation salt concentration while and/or submitting the mixture to protein-denaturation temperature allowing precipitation of the one or more denatured proteins;
(c) isolating the precipitated one or more proteins from the solvent in which they were precipitated, and resuspending them in a fresh buffered solvent, said step of resuspending with a fresh solvent comprising:
(c.I) Mixing the precipitated protein with one or more lipids dissolved in an organic solvent and wherein the ratio of the amount of protein and lipids is from 0.8:1 to 1:0.8;
(c.2) removing the organic solvent to obtain a dry film of denatured proteins and lipids
(c.3) suspending the dry film of step (c.2) with a buffered composition, optionally comprising one or more functional proteins, while agitating the mixture under a controlled temperature from 4° C. to 8° C. to obtain a protein-lipid nano- or microparticle comprising a cluster of assembled self-contained proteins and one or more types of lipids assembled with the self-contained proteins, said cluster optionally comprising one or more functional proteins embedded and/or adsorbed within the cluster of assembled self-contained proteins and lipids;
(c.4) separate the protein-lipid nano- and/or microparticle from the remaining buffered composition.

10. A protein nano- or microparticle as defined in claim 1, for use as a medicament.

11. The protein nano- or microparticle for use according to claim 10, which is for use in the treatment of a disease selected from the group consisting of cancer, an immune disease, neurodegenerative disease, and combinations thereof.

12. The protein nano- or microparticle for use according to claim 10, which is for use in the treatment of cancer.

13. The protein nano- or microparticle for use according to claim 12, wherein the cancer is colorectal cancer.

14. A pharmaceutical composition comprising a therapeutically effective amount of the protein a nano- or microparticle as defined in claim 1, together with
pharmaceutically acceptable excipients or carriers.

15. The pharmaceutical composition according to claim 14, which is for subcutaneous administration.


SEQUENCE LISTING (2 sequences)

SEQ ID NO: 1 - protein, 269 aa, Artificial
  Fusion protein
MRRWCYRKCYKGYCYRKCRGGSSRSSSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKHHHHHH

SEQ ID NO: 2 - protein, 269 aa, Artificial
  Fusion protein
MRRWCYRKCYKGYCYRKCRGGSSRSSRHRQPRGWEQLGGSPTGAEFLGDGGDVSFSTRGTQNWTVERLLQAHAQLEERGYVFVGYHGTFLEAAQSIVFGGVAARSQDLAAIWAGFYIAGDPALAYGYAQDQEPDAAGRIRNGALLRVYVPASSLPGFYRTSLTLAAPEAAGEVERLIGHPLPLALDAITGPEEEGGRLETILGWPLAERTVVIPSAIPTDPRNVGGDLDPSSIPDKEQAISALPDYASQPGKPPREDLKHHHHHHKDEL